SEQUENCE LISTING (78 sequences)

SEQ ID NO: 1 - DNA, 4139 bp, Homo sapiens
ccgctccacctctcaagcagccagcgcctgcctgaatctgttctgccccctccccacccatttcaccaccaccatgacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagctcggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaacggtggcagcagcctctcttacacaaacccagcagtggcagccgcttctgccaacttgtagggcacgtcgccgctgagctgagtggccagccagtgccattccactccactcaggttcttcaggccagagcccctgcaccctgtttgggctggtgagctgggagttcaggtgggctgctcacagcctccttcagaggccccaccaatttctcggacacttctcagtgtgtggaagctcatgtgggcccctgaggctcatgcctgggaagtgttgtgggggctcccaggaggactggcccagagagccctgagatagcggggatcctgaactggactgaataaaacgtggtctcccactg

SEQ ID NO: 2 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 3 - DNA, 1041 bp, Homo sapiens
atggcaatggtatcagaattcctcaagcaggcctggtttattgaaaatgaagagcaggaatatgttcaaactgtgaagtcatccaaaggtggtcccggatcagcggtgagcccctatcctaccttcaatccatcctcggatgtcgctgccttgcataaggccataatggttaaaggtgtggatgaagcaaccatcattgacattctaactaagcgaaacaatgcacagcgtcaacagatcaaagcagcatatctccaggaaacaggaaagcccctggatgaaacacttaagaaagcccttacaggtcaccttgaggaggttgttttagctctgctaaaaactccagcgcaatttgatgctgatgaacttcgtgctgccatgaagggccttggaactgatgaagatactctaattgagattttggcatcaagaactaacaaagaaatcagagacattaacagggtctacagagaggaactgaagagagatctggccaaagacataacctcagacacatctggagattttcggaacgctttgctttctcttgctaagggtgaccgatctgaggactttggtgtgaatgaagacttggctgattcagatgccagggccttgtatgaagcaggagaaaggagaaaggggacagacgtaaacgtgttcaataccatccttaccaccagaagctatccacaacttcgcagagtgtttcagaaatacaccaagtacagtaagcatgacatgaacaaagttctggacctggagttgaaaggtgacattgagaaatgcctcacagctatcgtgaagtgcgccacaagcaaaccagctttctttgcagagaagcttcatcaagccatgaaaggtgttggaactcgccataaggcattgatcaggattatggtttcccgttctgaaattgacatgaatgatatcaaagcattctatcagaagatgtatggtatctccctttgccaagccatcctggatgaaaccaaaggagattatgagaaaatcctggtggctctttgtggaggaaactaa

SEQ ID NO: 4 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 5 - protein, 167 aa, Mus musculus
PAQFDADELRGAMKGLGTDEDTLIEILTTRSNEQIREINRVYREELKRDLAKDITSDTSGDFRKALLALAKGDRCQDLSVNQDLADTDARALYEAGEIRKGTDVNVFTTILTSRSFPHLRRVFQNYGKYSQHDMNKALDLELKGDIEKCLTTIVKCATSTPAFFAEK

SEQ ID NO: 6 - protein, 7 aa, Homo sapiens
VSEFLKQ

SEQ ID NO: 7 - protein, 9 aa, Homo sapiens
QEYVQTVKS

SEQ ID NO: 8 - protein, 10 aa, Homo sapiens
PGSAVSPYPT

SEQ ID NO: 9 - protein, 16 aa, Homo sapiens
PSSDVAALHKAIMVKG

SEQ ID NO: 10 - protein, 9 aa, Homo sapiens
RQQIKAAYL

SEQ ID NO: 11 - protein, 19 aa, Homo sapiens
LKKALTGHLEEVVLALLKT

SEQ ID NO: 12 - protein, 20 aa, Homo sapiens
PDTRPAPGSTAPPAHGVTSA

SEQ ID NO: 13 - protein, 20 aa, Homo sapiens
PDNKPAPGSTAPPAHGVTSA

SEQ ID NO: 14 - protein, 24 aa, Homo sapiens
PTTTPPITTTTTVTPTPTPTGTQT

SEQ ID NO: 15 - protein, 17 aa, Homo sapiens
HSTPSFTSSITTTETTS

SEQ ID NO: 16 - protein, 16 aa, Homo sapiens
TSSASTGHATPLPVTD

SEQ ID NO: 17 - protein, 8 aa, Homo sapiens
TTSTTSAP

SEQ ID NO: 18 - protein, 29 aa, Homo sapiens
SSTPGTAHTLTMLTTTATTPTATGSTATP

SEQ ID NO: 19 - protein, 22 aa, Homo sapiens
TTAAPPTPSATTPAPPSSSAPG

SEQ ID NO: 20 - protein, 41 aa, Homo sapiens
TSCPRPLQEGTPGSRAAHALSRRGHRVHELPTSSPGGDTGF

SEQ ID NO: 21 - protein, 21 aa, Mus musculus
METDTLLLWVLLLWVPGSTGD

SEQ ID NO: 22 - DNA, 41 bp, Artificial Sequence
  Description of Artificial Sequence Synthetic primer
ccaccatggagacagacacactcctgctatgggtactgctg

SEQ ID NO: 23 - DNA, 37 bp, Artificial Sequence
  Description of Artificial Sequence Synthetic primer
tcctgctatgggtactgctgctctgggttccaggttc

SEQ ID NO: 24 - DNA, 33 bp, Artificial Sequence
  Description of Artificial Sequence Synthetic primer
tgctctgggttccaggttccactggtgacgatg

SEQ ID NO: 25 - DNA, 36 bp, Artificial Sequence
  Description of Artificial Sequence Synthetic primer
ggttccactggtgacgatgtcacctcggtcccagtc

SEQ ID NO: 26 - DNA, 31 bp, Artificial Sequence
  Description of Artificial Sequence Synthetic primer
gagctcgagattgtggactggaggggcggtg

SEQ ID NO: 27 - DNA, 54 bp, Artificial Sequence
  Description of Artificial Sequence Synthetic nucleotide construct
ccgctcgagaacgacgcacaagcaccaaaatcaaaggtcgaagaggaagtaaac

SEQ ID NO: 28 - DNA, 53 bp, Artificial Sequence
  Description of Artificial Sequence Synthetic primer
gcgggcccgcggccgccgctagtctagagagtttgagtaagccaaaagatgag

SEQ ID NO: 29 - protein, 10 aa, Artificial Sequence
  Description of Artificial Sequence Synthetic peptide
LENDAQAPKS

SEQ ID NO: 30 - DNA, 996 bp, Artificial Sequence
  Description of Artificial Sequence Synthetic nucleotide construct
atgtataggatgcaactgctgtcttgcattgctctgtctctggcactggtcactaactctgccaggatccacctctgggttccaggttccactggtgacgatgtcacctcggtcccagtcaccaggccagccctgggccccaccaccccgccagcccacgatgtcacctcagccccggacaacaagccagccccgggctccaccgcccccccagcccacggtgtcacctcggccccggacaacaggcccgccttgggctccaccgcccctccagtccacaatgtcaccctcgagaacgacgcacaagcagatatcaacgacgcacaagcaccaaaatcagtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 31 - DNA, 37 bp, Artificial Sequence
  Description of Artificial Sequence Synthetic primer
cgggatccacctctgggttccaggttccactggtgac

SEQ ID NO: 32 - DNA, 34 bp, Artificial Sequence
  Description of Artificial Sequence Synthetic primer
gccgcgatatctgcttgtgcgtcgttctcgaggg

SEQ ID NO: 33 - DNA, 35 bp, Artificial Sequence
  Description of Artificial Sequence Synthetic primer
gcgatatcaacgacgcacaagcaccaaaatcagtg

SEQ ID NO: 34 - DNA, 43 bp, Artificial Sequence
  Description of Artificial Sequence Synthetic primer
cgggaattctgctctagatcagagtttgagtaagccaaaggac

SEQ ID NO: 35 - DNA, 36 bp, Artificial Sequence
  Description of Artificial Sequence Synthetic primer
aaagccatcatgtataggatgcaactgctgtcttgc

SEQ ID NO: 36 - DNA, 43 bp, Artificial Sequence; identical to SEQ ID NO: 34
  Description of Artificial Sequence Synthetic primer

SEQ ID NO: 37 - DNA, 62 bp, Artificial Sequence
  Description of Artificial Sequence Synthetic primer
aacaagctcattcagttcctgatctcactggtgggatccaacgacgcacaagcaccaaaatc

SEQ ID NO: 38 - DNA, 60 bp, Artificial Sequence
  Description of Artificial Sequence Synthetic primer
agccttcggcagaagcatgcccagcaacagaaagtcgtcaacaagctcattcagttcctg

SEQ ID NO: 39 - DNA, 46 bp, Artificial Sequence
  Description of Artificial Sequence Synthetic primer
aatgaggctctgtggcgggaggtggccagccttcggcagaagcatg

SEQ ID NO: 40 - DNA, 61 bp, Artificial Sequence
  Description of Artificial Sequence Synthetic primer
gatatcctcaggctcgagaacgacgcacaagcaccaaaagagaatgaggctctgtggcggg

SEQ ID NO: 41 - protein, 53 aa, Artificial Sequence
  Description of Artificial Sequence Synthetic peptide
LENDAQAPKENEALWREVASFRQKHAQQQKVVNKLIQFLISLVGSNDAQAPKS

SEQ ID NO: 42 - protein, 9 aa, Artificial Sequence
  Description of Artificial Sequence Synthetic peptide
LENDAQAPK

SEQ ID NO: 43 - protein, 8 aa, Artificial Sequence
  Description of Artificial Sequence Synthetic peptide
NDAQAPKS

SEQ ID NO: 44 - DNA, 51 bp, Artificial Sequence
  Description of Artificial Sequence Synthetic primer
atggtgatgatgaccggtacggagtttgagtaagccaaaagatgagaagcc

SEQ ID NO: 45 - DNA, 49 bp, Artificial Sequence
  Description of Artificial Sequence Synthetic primer
gtgctctagatcagaattcatggtgatggtgatgatgaccggtacggag

SEQ ID NO: 46 - DNA, 57 bp, Artificial Sequence
  Description of Artificial Sequence Synthetic nucleotide construct
ccgctcgagaacgacgcacaagcaccaaaatcagtgtatcttcatagaaggttggac

SEQ ID NO: 47 - DNA, 34 bp, Artificial Sequence
  Description of Artificial Sequence Synthetic primer
ccctctagatcagagtttgagtaagccaaaggac

SEQ ID NO: 48 - protein, 26 aa, Homo sapiens
MATGSRTSLLLAFGLLCLPWLQEGSA

SEQ ID NO: 49 - protein, 98 aa, Human papillomavirus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 50 - DNA, 30 bp, Artificial Sequence
  Description of Artificial Sequence Synthetic primer
ccctctagaatcagagtttcactaagccaa

SEQ ID NO: 51 - DNA, 30 bp, Artificial Sequence
  Description of Artificial Sequence Synthetic primer
atttgcggccgctgtaatcatgcatggaga

SEQ ID NO: 52 - DNA, 29 bp, Artificial Sequence
  Description of Artificial Sequence Synthetic primer
ccctcgagttatggtttctgagaacagat

SEQ ID NO: 53 - DNA, 91 bp, Artificial Sequence
  Description of Artificial Sequence Synthetic oligonucleotide
gatctccaccatggctacaggctcccggacgtccctgctcctggcttttggcctgctctgcctgccctggcttcaagagggcagtgccggc

SEQ ID NO: 54 - DNA, 91 bp, Artificial Sequence
  Description of Artificial Sequence Synthetic oligonucleotide
aggtggtaccgatgtccgagggcctgcagggacgaggaccgaaaaccggacgagacggacgggaccgaagttctcccgtcacggccgccgg

SEQ ID NO: 55 - DNA, 36 bp, Artificial Sequence
  Description of Artificial Sequence Synthetic primer
tgctctgggttccaggttccactggtgacatgcatg

SEQ ID NO: 56 - DNA, 44 bp, Artificial Sequence
  Description of Artificial Sequence Synthetic primer
tgggttccaggttccactggtgacatgcatggagatacacctac

SEQ ID NO: 57 - DNA, 34 bp, Artificial Sequence
  Description of Artificial Sequence Synthetic primer
ccgctcgagtggtttctgagaacagatggggcac

SEQ ID NO: 58 - DNA, 58 bp, Artificial Sequence
  Description of Artificial Sequence Synthetic primer
ccgctcgagaacgacgcacaagcaccaaaaagcaaggtcgaagaggaagtaaaccttc

SEQ ID NO: 59 - DNA, 46 bp, Artificial Sequence
  Description of Artificial Sequence Synthetic primer
cgcgccgcgcgctagtctagagagtttgagtaagccaaaagatgag

SEQ ID NO: 60 - DNA, 47 bp, Artificial Sequence
  Description of Artificial Sequence Synthetic primer
gagacctcgagcagtcagcatgatagaaacatacagccaaccttccc

SEQ ID NO: 61 - DNA, 49 bp, Artificial Sequence
  Description of Artificial Sequence Synthetic primer
cgcgccgcgcgcccctctagatcagagtttgagtaagccaaaagatgag

SEQ ID NO: 62 - DNA, 35 bp, Artificial Sequence
  Description of Artificial Sequence Synthetic primer
tgctctgggttccaggttccactggtgacgaactc

SEQ ID NO: 63 - DNA, 38 bp, Artificial Sequence
  Description of Artificial Sequence Synthetic primer
tccactggtgacgaactcacctacctgcccaccaatgc

SEQ ID NO: 64 - DNA, 34 bp, Artificial Sequence
  Description of Artificial Sequence Synthetic primer
ggagctcgagggctgggtccccatcaaagctctc

SEQ ID NO: 65 - protein, 9 aa, Artificial Sequence
  Description of Artificial Sequence Synthetic peptide
KIFGSLAFL

SEQ ID NO: 66 - protein, 10 aa, Artificial Sequence
  Description of Artificial Sequence Synthetic peptide
ELTYLPTNAS

SEQ ID NO: 67 - protein, 23 aa, Artificial Sequence
  Description of Artificial Sequence Synthetic peptide
PLHNQEVTAEDGTQRCEKCSKPC

SEQ ID NO: 68 - DNA, 35 bp, Artificial Sequence
  Description of Artificial Sequence Synthetic primer
tgctctgggttccaggttccactggtgacccagct

SEQ ID NO: 69 - DNA, 34 bp, Artificial Sequence
  Description of Artificial Sequence Synthetic primer
tccactggtgacccagctcagtttgatgcagatg

SEQ ID NO: 70 - DNA, 35 bp, Artificial Sequence
  Description of Artificial Sequence Synthetic primer
ggagctcgagcttctcggcaaagaaagctggagtg

SEQ ID NO: 71 - DNA, 51 bp, Artificial Sequence
  Description of Artificial Sequence Synthetic primer
aaccatcactcttctggtagatctccagctcagtttgatgcagatgaactc

SEQ ID NO: 72 - DNA, 46 bp, Artificial Sequence
  Description of Artificial Sequence Synthetic primer
ccgctcgaggcagatatctcagagtttgagtaagccaaaagatgag

SEQ ID NO: 73 - DNA, 42 bp, Artificial Sequence
  Description of Artificial Sequence Synthetic primer
ccgctggaggcagatatcggctgggtccccatcaaagctctc

SEQ ID NO: 74 - DNA, 33 bp, Artificial Sequence
  Description of Artificial Sequence Synthetic primer
gcagatatcgcaatggtatcagaattcctcaag

SEQ ID NO: 75 - DNA, 35 bp, Artificial Sequence
  Description of Artificial Sequence Synthetic primer
ccgctcgaggagtttttagcagagctaaaacaacc

SEQ ID NO: 76 - DNA, 35 bp, Artificial Sequence
  Description of Artificial Sequence Synthetic primer
tgctctgggttccaggttccactggtgacgcaatg

SEQ ID NO: 77 - DNA, 36 bp, Artificial Sequence
  Description of Artificial Sequence Synthetic primer
tccactggtgacgcaatggtatcagaattcctcaag

SEQ ID NO: 78 - DNA, 37 bp, Artificial Sequence
  Description of Artificial Sequence Synthetic primer
cggagctcgaggagtttttagcagagctaaaacaacc

(19) United States
(12) Patent Application Publication
(10) Pub. No.: US 2006/0286074 A1
(43) Pub. Date: Dec. 21, 2006

(54) Methods for immunotherapy of cancer

(76) Inventors: Yucheng Tang, San Diego, CA (US); Albert Deisseroth, San Diego, CA (US)

Correspondence Address: FOLEY & LARDNER LLP, P.O. BOX 80278, SAN DIEGO, CA, 92138-0278, US

(21) Appl. No.: 11/441,822

(22) Filed: May 26, 2006

Related U.S. Application Data
(60) Provisional application No. 60/686,534, filed on May 31, 2005.
(60) Provisional application No. 60/795,686, filed on Apr. 28, 2006.

(51) Int. Cl. A61K 48/00; C07K 14/47; C07H 21/04; C12N 15/861

(52) U.S. Cl. 424/93.2; 536/23.5; 435/456; 435/325; 530/350

(57) ABSTRACT

Provided are methods of generating an immune response to an antigen specifically associated with tumor vascular endothelial cells (TVECA). The method comprises administering to an individual an expression vector encoding the TVECA. The vector comprises a transcription unit encoding a secretable fusion protein, the fusion protein containing a TVECA and CD40 ligand. In other methods, administration of a fusion protein containing the TVECA and CD40 ligand is used to enhance the immune response above that obtained by vector administration alone. Further methods comprise the combination therapy using an expression vector encoding a secretable TVECA fusion protein and a tumor antigen vaccine.

### CROSS-REFERENCE TO RELATED APPLICATIONS

This application claims the benefit of priority under 35 U.S.C. § 119(e) to U.S. Provisional Application Ser. No. 60/686,534 filed May 31, 2005 and U.S. Provisional Application Ser. No. 60/795,686 filed Apr. 28, 2006, both of which are incorporated by reference herein in their entirety including all figures and tables.

### TECHNICAL FIELD

The present invention relates generally to the field of vaccines. In particular, the present invention relates to the use vaccines in the treatment of cancer.

### BACKGROUND ART

The following discussion of the background of the invention is merely provided to aid the reader in understanding the invention and is not admitted to describe or constitute prior art to the present invention.

The activation of antigen presenting cells (APCs), which includes the dendritic cells (DCs), followed by loading of antigen presenting cells with relevant antigens is a requisite step in the generation of a T cell dependent immune response against cancer cells. Once activated and loaded with tumor antigens, APCs migrate to regional lymph nodes (LNs) to present antigens to T cells. Very commonly, these APCs express insufficient amounts of surface activation molecules which are required for optimal activation and expansion of T cell clones competent to recognize tumor antigens. See Shortman, et al., Stem Cells 15:409-419, 1997.

Antigen presentation to naive T cells, in the absence of costimulatory molecule expression on the surface of the APC, leads to anergy of the T cells. See Steinbrink, et al. Blood 99: 2468-2476, 2002. Moreover, cross-presentation by DCs without CD4⁺ T cell help also results in peripheral deletion of Ag-specific T cells in regional LNs. See Kusuhara, et al., Eur J Immunol 32:1035-1043, 2002. In contrast, in the presence of CD4⁺ T cell help, DCs acquire functional ability to cross-prime T cells, resulting in clonal expansion of effector T cells. See Gunzer, et al., Semin Immunol 13:291-302, 2001. This CD4⁺ T cell help can be replaced with CD40-CD40 ligand (CD40L) interactions. See Luft, et al. Int Immunol 14:367-380, 2002. CD40L is a 33-kDa type II membrane protein and a member of the TNF gene family and is transiently expressed on CD4⁺ T cells after TCR engagement. See Skov, et al. J. Immunol. 164: 3500-3505, 2000.

The ability of DCs to generate anti-tumor immune responses in vivo has been documented in a number of animal tumor models. See Paglia, et al. J Exp Med 183: 317-322, 1996; Zitvogel, et al., J Exp Med. 183: 87-97, 1996. However, it is difficult to ensure that the antigen presenting cells express appropriate adhesion molecules and chemokine receptors to attract DCs to secondary lymphoid organs for priming T cells. See Fong, et al. J Immunol. 166: 4254-4259, 2001; Markowicz, et al. J Clin Invest. 85: 955-961, 1990; Hsu, et al. Nat Med. 2: 52-58, 1996; Nestle, et al. Nat Med. 4: 328-332, 1998; Murphy, et al., Prostate 38: 73-78, 1999; Dhodapkar, et al. J Clin Invest. 104: 173-180, 1999.

Vaccines have been described that include an expression vector encoding a fusion protein that includes an antigen fused to CD40 ligand. See, e.g., PCT/US03/36237 filed Nov. 12, 2003 entitled “adenoviral vector vaccine; and U.S. Patent Application Publication US 2005-0226888 (application Ser. No. 11/009,533) titled “Methods for Generating Immunity to Antigen,” filed Dec. 10, 2004.

### SUMMARY OF THE INVENTION

The methods and compositions of the invention are used to elicit an immune response to tumor vasculature. These methods and compositions are advantageous in several regards. First, by directing the immune response to the tumor vasculature, circulating antigen-specific effector T cells and antibodies have immediate access to this target tissue which directly faces the blood supply. In contrast, immune responses directed to the tumor cells must traverse the tumor vasculature and then penetrate the tumor to have effect. By directing the immune response to endothelial antigens that are associated solely with tumor vascular endothelial cells or expressed in higher amounts on tumor vascular endothelial cells, the impact on normal vascular endothelium is eliminated or reduced to acceptable levels. Moreover, the target endothelial cells of the tumor being genetically stable compared to the tumor cells are less likely to exhibit “immunological escape” (i.e. to modify the cell surface phenotype to avoid an immune response). Also, destruction vascular endothelial cells has the potential of destroying many more tumor cells by depriving the tumor of blood supply.

In a first aspect, the invention provides vaccines for generating an immune response against a tumor vascular endothelial cell antigen (“TVECA”). An immune response to the TVECA is achieved by administering an expression vector encoding a secretable fusion protein which includes a TVECA and CD40 ligand. The resulting immune response suppresses tumor growth and/or diminishes tumor size by destroying tumor vasculature, thereby depriving the tumor of blood supply.

The term “tumor vascular endothelial cell antigen” (TVECA) as used herein refers to an antigen that is selectively associated with the luminal membrane of tumor vascular endothelial cells. Exemplary TVECAs include annexin A1, annexin A8, VEGF R1, endosialin, and Tie2, markers which have been shown to be expressed at higher levels in the endothelial cells of tumor vessels as compared with the endothelial cells of the vessels of normal tissues (Oh et al., Nature 429:629-35, 2004; Christian et al., J Biol Chem 276(10):7408-14, 2001; Tanaka et al., Hepatology 35:861-7, 2002). “TVECA” as used herein may be a full length precursor or mature TVECA or may be a fragment of a TVECA provided the fragment forms at least one antigenic determinant that is capable of eliciting an immune response a described herein.

“Selectively associated” as used herein means that the antigen is unique to tumor endothelial versus normal vascular endothelial cells or is expressed in higher amounts on tumor endothelial cells versus normal vascular endothelial cells. The antigen may be an integral membrane protein that is luminally expressed or may be an extracellular antigen that is luminally associated. In either case, the antigen should be associated with the tumor vascular endothelial cells on its luminal side, thereby providing direct access to circulating antibodies and T cells. In preferred embodiments, TVECAs are uniquely expressed in tumor vascular endothelial cells and are not expressed in normal tissue (e.g. normal vascular endothelial cells). In other embodiments, TVECAs are expressed at such low levels in normal tissue (e.g. normal vascular endothelial cells) that such tissue is not significantly affected by the vaccine or subsequent immune response.

In another aspect, an immune response to the TVECA is elicited by administering an expression vector encoding the TVECA-CD40 ligand fusion protein and the fusion protein. The fusion protein is administered before, concurrently, or after administration of the vector. Preferably, the fusion protein is administered after the vector. The sequence of the TVECA encoded by the vector and that present in the fusion protein may be identical or may be different. If different, the two preferably have at least one antigenic determinant in common.

In one approach, the sequence encoding the TVECA in the fusion protein transcription unit is 5′ to sequence encoding the CD40 ligand. In another approach, the sequence encoding the CD40 ligand in the fusion protein transcription unit is 5′ to sequence encoding the TVECA. In a preferred embodiment, the CD40 ligand lacks all or a portion of its transmembrane domain.

Further provided herein are methods of treating an individual with cancer that expresses a TVECA by administering an expression vector encoding a secretable fusion protein which includes a TVECA and CD40 ligand. In some embodiments, the expression vector and the fusion protein encoded thereby are administered to the individual. The fusion protein may be administered before, concurrently or after administration of the vector. Preferably, the fusion protein is administered after the vector.

In preferred embodiments, the expression vector in any of the above methods may be a viral expression vector or a non-viral expression vector; the expression vector may be an adenoviral vector; the vector may be advantageously administered subcutaneously; the vector may be administered on a subsequent occasion(s) to increase the immune response; a signal sequence may be placed upstream of the fusion protein in the vector for secretion of the fusion protein; the transcription unit of the vector may include sequence that encodes a linker between the TVECA and the CD40 ligand; suitable linkers may vary in length and composition; the expression vector may include a human cytomegalovirus promoter/enhancer for controlling transcription of the transcription unit; and the CD40 ligand may be a human CD40 ligand.

In a further aspect, the invention methods of immunizing against a TVECA may be combined with immunization against a tumor antigen. Various approaches may be used for this purpose. For example, a single vector may be used that encodes both a secretable fusion protein comprising a TVECA and CD40 ligand and a secretable fusion protein that comprises a tumor antigen and CD40 ligand. Another single vector approach is to use a vector that encodes a soluble fusion protein that comprises TVECA, tumor antigen and CD40L. In this instance, the antigen portion of the soluble fusion protein is a chimeric antigen that includes both a TVECA and a tumor antigen. The vector encoding the chimeric antigen may be constructed so that the sequence encoding the TVECA may be upstream or downstream of sequence encoding the tumor antigen. Thus, in the resulting fusion protein, the TVECA may be carboxy terminal or amino terminal to the tumor antigen. The vector may encode a linker sequence between the two antigens. Separate vectors encoding only one of the secretable fusion proteins may be used. In yet another approach, the invention methods of immunizing against a TVECA may be combined with methods of immunizing with a tumor antigen that do not involve use of CD40 ligand.

Tumor antigens include both tumor associated antigens and tumor specific antigens. The term “tumor associated antigen” (TAA) as used herein refers to a protein which is present on tumor cells, and on normal cells during fetal life (onco-fetal antigen), after birth in selected organs, or on many normal cells, but at much lower concentration than on tumor cells. A variety of TAA have been described. An exemplary TAA is a mucin such as MUC1, described in further detail below, or the HER2 (neu) antigen, also described below.

The term “tumor specific antigen” (TSA) (aka. “tumor-specific transplantation antigen or TSTA) as used herein refers to a protein present on a tumor cell but absent from all normal cells. TSAs usually appear when an infecting virus has caused the cell to become immortal and to express a viral antigen(s). An exemplary viral TSA is the E6 or E7 proteins of human papilloma virus (HPV) type 16. HPV can cause a variety of epithelial lesions of the skin and genital tract. HPV related diseases of the genital tract constitute the second leading cause of cancer death among women in the world. These include genital warts, cervical intraepithelial neoplasia (CIN) and cancer of the cervix. The HPV type most commonly associated with high grade CIN and cervical cancer is HPV type 16. The majority of cervical cancers express the non-structural HPV16-derived gene products E6 and E7 oncoproteins. In HPV-induced cervical cancer model, the E6/E7 oncoproteins are required for maintenance of the malignant phenotype and their expression correlates with the transforming potential of HPV16.

TSAs not induced by viruses can be idiotypes of the immunoglobulin on B cell lymphomas or the T cell receptor (TCR) on T cell lymphomas.

In preferred embodiments, the tumor antigen is the E6 or E7 protein of human papilloma virus; a mucin antigen, which may be selected from the group consisting of MUC1, MUC2, MUC3A, MUC3B, MUC4, MUC5AC, MUC5B, MUC6, MUC7, MUC8, MUC9, MUC12, MUC13, MUC15, and MUC16; the MUC1 mucin antigen; or a human epidermal growth factor (EGF) receptor or related antigen (e.g., HER1, HER2, HER3 and HER4).

“Tumor antigen” as used herein may be a full length mature tumor antigen or may be a fragment of a tumor antigen provided the fragment forms at least one antigenic determinant that is capable of eliciting an immune response a described herein. An antigenic fragment may be determined by testing the immune response with portions of the molecule such as are predicted to carry an epitope using well known computer alogorithms (e.g. Hopp and Woods hydrophobicity analysis).

Using the methods of the invention, immunity against the TVECA may be long lasting and involve generation of cytotoxic CD8⁺ T cells against TVECA expressing cells and the production of antibody to the TVECA.

The use of a transcription unit encoding a “secretable fusion protein,” wherein the fusion protein comprises the TVECA and CD40 ligand means that the fusion protein is capable of being secreted by a cell containing the expression vector in substantial amounts. “Substantial” as used in this instance means that the amount of fusion protein that can be secreted from infected cells in an individual is sufficient to generate an immune response for the purposes described herein. Generally, a substantial amount is where at least 20% of the fusion protein produced by a cell is secreted by the cell.

For example, in the case of a TVECA, the transmembrane domain, if present, is generally about 20-30 amino acids in length and functions to anchor TVECA or a fragment thereof in the cell membrane. A TVECA missing substantially all of the transmembrane is one where the domain comprises 6 residues or less, more preferably less than about 4 residues of sequence, even more preferably less than about 2 residues of sequence and most preferably 1 residue or less of sequence. Any transmembrane sequence that is present may be at one end of the domain or may be divided between both ends. In a preferred embodiment, the vaccine vector transcription unit encodes a secretable form of a TVECA lacking the entire transmembrane domain. Likewise, in the portion of the fusion protein relating to CD40 ligand, the secretable form of the fusion protein is one where the CD40L is missing all or substantially all of the transmembrane domain rendering CD40. The transmembrane domain of CD40L which contains about 24 amino acids in length, functions to anchor CD40 ligand in the cell membrane. CD40L from which all of the transmembrane domain has been deleted is CD40 ligand lacking residues 23-46. CD40 ligand missing substantially all of the transmembrane is one that comprises 6 residues or less, more preferably less than about 4 residues of sequence, even more preferably less than about 2 residues of sequence and most preferably 1 residue or less of sequence. Any transmembrane sequence that is present from the CD40L may be at one end of the domain or may be divided between both ends. In a preferred embodiment, the vaccine vector transcription unit encodes a secretable form of a TVECA wherein CD40L is lacking the entire transmembrane domain.

In designing a transcription unit encoding a “secretable fusion protein,” wherein the fusion protein comprises the TVECA and CD40 ligand, one may take into account the amounts of transmembrane sequence present from the TVECA and CD40L. In a preferred embodiment, there is no transmembrane domain sequence for either TVECA or CD40L of the fusion protein. The exact amount of transmembrane domain sequence that may be used in the TVECA or CD40L portions of the fusion protein can be determined by evaluating the amount of secreted fusion protein using routine methods. In some embodiments, there may be some transmembrane domain sequence for TVECA or CD40L but not both. In other embodiments, there may be transmembrane domain sequence for both TVECA and CD40L of the fusion protein. In general, the amount of transmembrane domain from TVECA or CD40L that is present in the fusion protein is 10% or less of the native transmembrane domain.

In yet another aspect, the invention provides nucleic acids encoding a fusion protein comprising a TVECA and CD40 ligand, the fusion protein encoded thereby, and a vector comprising such nucleic acids, along the lines described herein for generating an immune response in an individual against tumor vasculature.

Abbreviations used herein include “Ad” (adenoviral); “sig” (signal sequence); “sp” (spacer or linker sequence); and “ecd” (extracellular domain).

These and other embodiments are described in detail below.

### BRIEF DESCRIPTION OF THE DRAWINGS

FIG. 1 shows the nucleotide sequence encoding human MUC1 (GenBank ID No. g189598, SEQ ID NO:1).

FIG. 2 shows the amino acid sequence of human MUC1 (SEQ ID NO:2). The extracellular domain is underlined.

FIG. 3 shows the nucleotide sequence encoding human annexin A1 (GenBank Accession No. NM_{—}000700, SEQ ID NO:3).

FIG. 4 shows the amino acid sequence of human annexin A1 (GenBank Accession No. NP_{—}000691, SEQ ID NO:4).

FIG. 5 demonstrates suppression of tumor growth by subcutaneous injection of Ad-sig-AnxA1/ecdCD40L vector.

FIG. 6 demonstrates the presence of antibodies against annexin A1 in serum.

FIG. 7 demonstrates the inhibition of breast cancer growth in a ratHer2/neu transgenic model by a combination of vaccines against annexin A1 and Her2/neu antigens. The groups tested are as follows: Ad-sig-Her2neu/ecdCD40L (diamond); PBS control (triangle); Ad-sig-AnnexinA1/ecdCD40L (circle); and Ad-sig-AnnexinA1/ecdCD40L plus Ad-sig-Her2neu/ecdCD40L (square). Shown is the tumor volume for each group 14, 21, 36, and 43 days after the tumor challenge.

FIG. 8 demonstrates that the percentage of tumor-free mice following combination of vaccines against annexin A1 and Her2/neu antigens is higher than for either vaccine alone. The groups tested are as follows: Ad-sig-Her2neu/ecdCD40L (diamond); PBS control (triangle); Ad-sig-AnnexinA1/ecdCD40L (circle); and Ad-sig-AnnexinA1/ecdCD40L plus Ad-sig-Her2neu/ecdCD40L (square). Shown is the percentage of tumor-free mice for each group 14, 21, 36, and 43 days after the tumor challenge.

### DETAILED DESCRIPTION OF THE INVENTION

In accordance with one aspect of the invention, a method is provided for generating an immune response in an individual against a TVECA using an expression vector. The vector includes a transcription unit encoding a secretable fusion protein containing the TVECA and CD40 ligand. In one embodiment, the transcription unit includes from the amino terminus, a secretory signal sequence, the TVECA, a linker, and a secretable form of CD40 ligand. In preferred embodiments, the secretable form of CD40 ligand lacks all or substantially all of its transmembrane domain. In one approach, the individual is administered the vector on one or more occasions to generate an immune response.

In another approach, the fusion protein is also administered in an effective amount before, concurrently, or after administration of vector to boost the immune response to the antigen above that obtained with vector administration alone. In a preferred embodiment, the fusion protein is administered after administration of the expression vector.

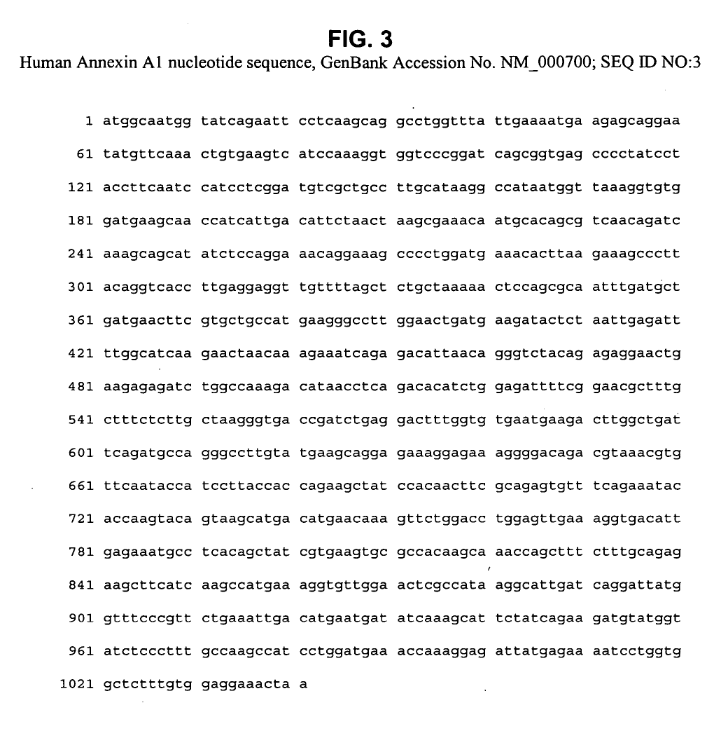

The term “in an effective amount” in reference to administering the fusion protein is an amount that generates an increased immune response over that obtained using the expression vector alone. A time interval between administrations is generally required for optimal results. An increase in the immune response may be measured as an increase in T cell activity or antibody production (see e.g., FIGS. 3-5 of U.S. Patent Application Publication US 2005-0226888 (application Ser. No. 11/009,533) titled “Methods for Generating Immunity to Antigen”). Generally, at least one week between vector administration and protein boosting is effective although a shorter interval may be possible. An effective spacing between administrations may be from 1 week to 12 weeks or even longer. Multiple boosts may be given which may be separated by from 1-12 weeks or even longer periods of time.

The use of the fusion protein to boost the immune response avoids having to repetitively administer the expression vector which might generate hypersensitivitiy to multiple injections. The antigen portion of the fusion protein is preferably the fusion protein which is encoded by the transcription unit of the expression vector used in the initial administration. However, the antigen portion of the fusion protein may differ from the encoded antigen provided that there is at least one shared antigenic determinant or epitope common to the antigen of the expression vector and that of the fusion protein used for boosting.

The fusion protein may be prepared in a mammalian cell line system, which is complementary to the vector. For example, in the case of adenovirus, the cell line system can be 293 cells that contain the Early Region 1 (E1) gene and can support the propagation of the E1-substituted recombinant adenoviruses. When the adenoviral vectors infect the production cells, the viral vectors will propagate themselves following the viral replication cycles. However, the gene of interest that is carried by the viral vector in the expression cassette will express during the viral propagation process. This can be utilized for preparation of the fusion protein encoded by the vector in the same system for production of the vector. The production of both the vector and the fusion protein will take place simultaneously in the production system. The vector and protein thus produced can be further isolated and purified via different processes. Alternatively, the vector and fusion protein can be produced in different systems. For example, the vector may be produced as described above and the fusion protein may be produced using a bacterial cell expression system.

The vector or fusion protein may be administered parenterally, such as intravascularly, intravenously, intraarterially, intramuscularly, subcutaneously, or the like. Administration can also be orally, nasally, rectally, transdermally or inhalationally via an aerosol. The protein boost may be administered as a bolus, or slowly infused. The protein boost is preferably administered subcutaneously.

The fusion protein boost may be formulated with an adjuvant to enhance the resulting immune response. As used herein, the term “adjuvant” means a chemical that, when administered with the vaccine, enhances the immune response to the vaccine. An adjuvant is distinguished from a carrier protein in that the adjuvant is not chemically coupled to the immunogen or the antigen. Adjuvants are well known in the art and include, for example, mineral oil emulsions (U.S. Pat. No. 4,608,251, supra) such as Freund's complete or Freund's incomplete adjuvant (Freund, Adv. Tuberc. Res. 7:130 (1956); Calbiochem, San Diego Calif.), aluminum salts, especially aluminum hydroxide or ALHYDROGEL (approved for use in humans by the U.S. Food and Drug Administration), muramyl dipeptide (MDP) and its analogs such as [Thrl]-MDP (Byers and Allison, Vaccine 5:223 (1987)), monophosphoryl lipid A (Johnson et al., Rev. Infect. Dis. 9:S512 (1987)), and the like.

The fusion protein can be administered in a microencapsulated or a macroencapsulated form using methods well known in the art. Fusion protein can be encapsulated, for example, into liposomes (see, for example, Garcon and Six, J. Immunol. 146:3697 (1991)), into the inner capsid protein of bovine rotavirus (Redmond et al., Mol. Immunol. 28:269 (1991)) into immune stimulating molecules (ISCOMS) composed of saponins such as Quil A (Morein et al., Nature 308:457 (1984); Morein et al., Immunological Adjuvants and Vaccines (G. Gregoriadis al. eds.) pp. 153-162, Plenum Press, NY (1987)) or into controlled-release biodegradable microspheres composed, for example, of lactide-glycolide compolymers (O'Hagan et al., Immunology 73:239 (1991); O'Hagan et al., Vaccine 11:149 (1993)).

The fusion protein also can be adsorbed to the surface of lipid microspheres containing squalene or squalane emulsions prepared with a PLURONIC block-copolymer such as L-121 and stabilized with a detergent such as TWEEN 80 (see Allison and Byers, Vaccines: New Approaches to Immunological Problems (R. Ellis ed.) pp. 431-449, Butterworth-Hinemann, Stoneman N.Y. (1992)). A microencapsulated or a macroencapsulated fusion protein can also include an adjuvant.

The fusion protein also may be conjugated to a carrier or foreign molecule such as a carrier protein that is foreign to the individual to be administered the protein boost. Foreign proteins that activate the immune response and can be conjugated to a fusion protein as described herein include proteins or other molecules with molecular weights of at least about 20,000 Daltons, preferably at least about 40,000 Daltons and more preferably at least about 60,000 Daltons. Carrier proteins useful in the present invention include, for example, GST, hemocyanins such as from the keyhole limpet, serum albumin or cationized serum albumin, thyroglobulin, ovalbumin, various toxoid proteins such a tetanus toxoid or diptheria toxoid, immunoglobulins, heat shock proteins, and the like.

Methods to chemically couple one protein to another (carrier) protein are well known in the art and include, for example, conjugation by a water soluble carbodiimide such as 1-ethyl-3-(3dimethylaminopropyl)carbodiimide hydrochloride, conjugation by a homobifunctional cross-linker having, for example, NHS ester groups or sulfo-NHS ester analogs, conjugation by a heterobifunctional cross-linker having, for example, and NHS ester and a maleimide group such as sulfosuccinimidyl-4-(N-maleimidomethyl)cyclohexane-1-carboxylate and, conjugation with gluteraldehyde (see, for example, Hermanson, Bioconjugate Techniques, Academic Press, San Diego, Calif. (1996)); see, also, U.S. Pat. Nos. 4,608,251 and 4,161,519).

The term “vector” which contains a transcription unit (aka. “expression vector”) as used herein refers to viral and non-viral expression vectors that when administered in vivo can enter target cells and express an encoded protein. Viral vectors suitable for delivery in vivo and expression of an exogenous protein are well known and include adenoviral vectors, adeno-associated viral vectors, retroviral vectors, herpes simplex viral vectors, and the like. Viral vectors are preferably made replication defective in normal cells. See U.S. Pat. Nos. 6,669,942; 6,566,128; 6,794,188; 6,110,744; 6,133,029.

As used herein, the term “cells” is used expansively to encompass any living cells such as mammalian cells, plant cells, eukaryotic cells, prokaryotic cells, and the like.

The term “adenoviral expression vector” as used herein, refers to any vector from an adenovirus that includes exogenous DNA inserted into its genome which encodes a polypeptide. The vector must be capable of replicating and being packaged when any deficient essential genes are provided in trans. An adenoviral vector desirably contains at least a portion of each terminal repeat required to support the replication of the viral DNA, preferably at least about 90% of the full ITR sequence, and the DNA required to encapsidate the genome into a viral capsid. Many suitable adenoviral vectors have been described in the art. See U.S. Pat. Nos. 6,440,944 and 6,040,174 (replication defective E1 deleted vectors and specialized packaging cell lines). A preferred adenoviral expression vector is one that is replication defective in normal cells.

Adeno-associated viruses represent a class of small, single-stranded DNA viruses that can insert their genetic material at a specific site on chromosome 19. The preparation and use of adeno-associated viral vectors for gene delivery is described in U.S. Pat. No. 5,658,785.

Non-viral vectors for gene delivery comprise various types of expression vectors (e.g., plasmids) which are combined with lipids, proteins and other molecules (or combinations of thereof) in order to protect the DNA of the vector during delivery. Fusigenic non-viral particles can be constructed by combining viral fusion proteins with expression vectors as described. Kaneda, Curr Drug Targets (2003) 4(8):599-602. Reconstituted HVJ (hemagglutinating virus of Japan; Sendai virus)-liposomes can be used to deliver expression vectors or the vectors may be incorporated directly into inactivated HVJ particles without liposomes. See Kaneda, Curr Drug Targets (2003) 4(8):599-602. DMRIE/DOPE lipid mixture are useful a vehicle for non-viral expression vectors. See U.S. Pat. No. 6,147,055. Polycation-DNA complexes also may be used as a non-viral gene delivery vehicle. See Thomas et al., Appl Microbiol Biotechnol (2003) 62(1):27-34.

The term “transcription unit” as it is used herein in connection with an expression vector means a stretch of DNA that is transcribed as a single, continuous mRNA strand by RNA polymerase, and includes the signals for initiation and termination of transcription. For example, in one embodiment, a transcription unit of the invention includes nucleic acid that encodes from 5′ to 3,′ a secretory signal sequence, an antigen and CD40 ligand, in the same reading frame. The transcription unit is in operable linkage with transcriptional and/or translational expression control elements such as a promoter and optionally any upstream or downstream enhancer element(s). A useful promoter/enhancer is the cytomegalovirus (CMV) immediate-early promoter/enhancer. See U.S. Pat. Nos. 5,849,522 and 6,218,140.

The term “secretory signal sequence” (aka. “signal sequence,” “signal peptide,” leader sequence,” or “leader peptide”) as used herein refers to a short peptide sequence, generally hydrophobic in charter, including about 20 to 30 amino acids which is synthesized at the N-terminus of a polypeptide and directs the polypeptide to the endoplasmic reticulum. The secretory signal sequence is generally cleaved upon translocation of the polypeptide into the endoplasmic reticulum. Eukaryotic secretory signal sequences are preferred for directing secretion of the exogenous gene product of the expression vector. A variety of suitable such sequences are well known in the art and include the secretory signal sequence of human growth hormone, immunoglobulin kappa chain, and the like. In some embodiments the endogenous tumor antigen signal sequence also may be used to direct secretion.

The term “antigen” as used herein refers broadly to any antigen to which an individual can generate an immune response. “Antigen” as used herein refers broadly to a molecule that contains at least one antigenic determinant to which the immune response may be directed. The immune response may be cell mediated or humoral or both.

As is well-known in the art, an antigen may be protein in nature, carbohydrate in nature, lipid in nature, nucleic acid in nature, or combinations of these biomolecules. As is well-known in the art, an antigen may be native, recombinant, or synthetic. For example, an antigen may include non-natural molecules such as polymers and the like. Antigens include self antigens and foreign antigens such as antigens produced by another animal or antigens from an infectious agent. Infectious agent antigens may be bacterial, viral, fungal, protozoan, and the like.

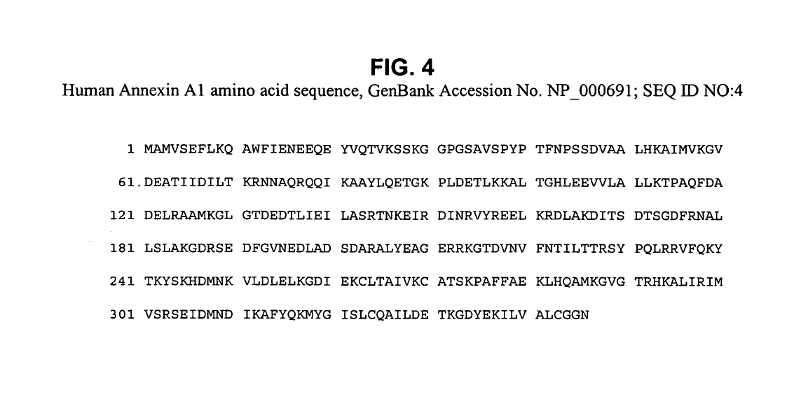

In preferred embodiments, the TVECA is annexin A1 (“AnxA1” or “AnnexA1”). Annexin A1 is a member of the annexin family of Ca²⁺/lipid-binding proteins. Annexin A1 is a cytsolic protein in normal cells, but appears at the surface of the luminal membrane of the tumor vascular endothelial cells. Annexins have a unique architecture that allows docking onto a membrane in a peripheral and reversible manner. The annexin core domain includes the conserved Ca²⁺- and membrane-binding module and consists of four annexin repeats, each of which is about 50-70 residues in length. The annexin core is highly helical and forms a compact, slightly curved disc having a convex surface, which harbors the Ca²⁺- and membrane-binding sites, and a concave surface, which points away from the membrane and is therefore available for interaction with other molecules. The N-terminal region precedes the core domain and is diverse in sequence and length. In vertebrates, 12 annexin subfamilies have been identified (A1-A11 and A13), which have different splice variants. Each subfamily has different N-terminal domains and differently positioned Ca²⁺-/membrane-binding sites within the core domain. An exemplary nucleotide sequence of human annexin A1 and the protein encoded thereby are set forth in FIG. 3 (GenBank Accession No. NM_{—}000700) and FIG. 4 (GenBank Accession No. NP_{—}000691), respectively. This protein consists of 346 amino acids and contains four conserved PFAM domains (i.e., annexin repeat domains), each consisting of about 65 amino acid residues at the following locations: residues 47-111, 117-183, 205-267, and 276-342.

In some embodiments, the TVECA may be native, recombinant, or synthetic annexin A1. In other embodiments, the TVECA may be an annexin A1 protein or fragment, or a nucleic acid encoding annexin A1 or an annexin A1 fragment. In some embodiments the antigen comprises the first annexin repeat (e.g. amino acids 47-111) (J. Biol. Chem. 266:6670-3, 1991). In other embodiments the antigen comprises the second, third, or fourth annexin repeat. In further embodiments more that one of the annexin repeats are combined to form the antigen. In such a case the antigen can comprise the intervening sequence between the repeats or the repeats can be adjoining in the expression vector. Additional amino acid residues may also be included on either end of the repeats. In one example, the annexin A1 antigen comprises amino acid residues 115-281 of mouse annexin A1 and has the following sequence:

| | |
|---|---|
| PAQFDADELRGAMKGLGTDEDTLIEILTTRSNEQIRE | (SEQ ID NO:5) |
| INRVYREELKRDLAKDITSDTSGDFRKALLALAKGDR | |
| CQDLSVNQDLADTDARALYEAGEIRKGTDVNVFTTIL | |
| TSRSFPHLRRVFQNYGKYSQHDMNKALDLELKGDIEK | |
| CLTTIVKCATSTPAFFAEK. | |

Further examples of annexin A1 antigens may be predicted by one of skill in the art using any of a number of computer programs for predicting antigenic determinants (e.g., Predicted Antigenic Peptides, CVC Bioinformatics from The Molecular Immunology Foundation). The table below constains exemplary sequences from human annexin A1 (GenBank Accession No. NP_{—}000691) that are predicted to be antigenic.

| | | | |
|---|---|---|---|
| SEQ ID | Start | | End |
| NO: | Position | Sequence | Position |

| | | | |
|---|---|---|---|
| 6 | 4 | VSEFLKQ | 10 |
| 7 | 19 | QEYVQTVKS | 27 |
| 8 | 32 | PGSAVSPYPT | 41 |
| 9 | 44 | PSSDVAALHKAIMVKG | 59 |
| 10 | 77 | RQQIKAAYL | 85 |
| 11 | 96 | LKKALTGHLEEVVLALLKT | 114 |

The annexin A1 antigen may include any combination of the above predicted antigenic sequences. In one example, the annexin A1 comprises amino acids 1-114 of human annexin A1 and therefore includes all of the above predicted antigenic sequences. Further annexin A1 antigens can include variants or splice variants of annexin A1, or annexin A1 having post-translational modifications.

In other embodiments, the TVECA is annexin A8 (AnxA8). This protein is an anticoagulant protein that acts as an indirect inhibitor of the thromboplastin-specific complex, which is involved in the blood coagulation cascade. An exemplary amino acid sequence of annexin A8 can be found in Swiss-Prot Accession No. P13928. This protein consists of 327 amino acids. This annexin A8 contains four annexin repeats, each consisting of 61 amino acid residues at the following locations: residues 30-90, 102-162, 187-247, and 262-322. The invention methods can use native, recombinant, or synthetic annexin A8. The invention methods can use an annexin A8 protein or fragment, or a nucleic acid encoding annexin A8 or an annexin A8 fragment. The invention methods can use variants or splice variants of annexin A8, or annexin A8 having post-translational modifications.

In other embodiments, the TVECA is vascular endothelial growth factor receptor-1 (VEGF R1). VEGF R1 is a member of the VEGFR family of receptor tyrosine kinases (RTK), which have been implicated in the process of angiogenesis. Angiogenesis involves endothelial cell differentiation, proliferation, migration and cord formation, which lead to tubulogenesis to form vessels. VEGF R1 is a kinase-impaired RTK. VEGFR-1 regulates angiogenesis by mechanisms that involve ligand-trapping, receptor homo- and heterodimerization and is required for normal development and angiogenesis.

An exemplary amino acid sequence of human VEGF R1 can be found in Swiss-Prot Accession No. P17948. This protein consists of 1338 amino acids and comprises the following domains: a signal peptide (amino acid residues 1-26), an extracellular domain (amino acid residues 27-758), a transmembrane domain (amino acid residues 759-780), and a cytoplasmic domain (amino acid residues 781-1338). In some embodiments the TVECA may be native, recombinant, or synthetic VEGF R1. In other embodiments, the TVECA may be a VEGF R1 protein or fragment, or a nucleic acid encoding VEGF R1 or a VEGF R1 fragment. Preferred fragments of VEGF R1 include all or a portion of the extracellular domain. The invention methods can use variants or splice variants of VEGF R1, or VEGF R1 having post-translational modifications.

In other embodiments, the TVECA is endosialin. Endosialin is a cell surface glycoprotein that is expressed in tumor vasculature endothelium of many types of human cancer, but has not been detected or has been detected at low levels in the endothelial cells or other cell types of many normal tissues. Endosialin is a type I membrane protein consisting of 757 amino acids. Sequence analysis indicates that this protein consists of a signal peptide, five globular extracellular domains (i.e., a C-type lectin domain, a domain with similarity to the Sushi/ccp/scr pattern, and three EGF repeats), a mucin-like region, a transmembrane domain, and a cytoplasmic tail (Christian et al., J Biol Chem 276(10):7408-14, 2001).

An exemplary amino acid sequence of human endosialin can be found in Swiss-Prot Accession No. Q9HCUO. This protein consists of 757 amino acids and comprises the following domains: a signal peptide (amino acid residues 1-17), an extracellular domain (amino acid residues 18-687), a transmembrane domain (amino acid residues 688-708), and a cytoplasmic domain (amino acid residues 709-757). In some embodiments the TVECA may be native, recombinant, or synthetic endosialin. In other embodiments, the TVECA may be an endosialin protein or fragment or a nucleic acid encoding an endosialin or an endosialin fragment. Preferred fragments of endosialin include all or a portion of the extracellular domain. The invention methods can use variants or splice variants of an endosialin or an endosialin having post-translational modifications.

In other embodiments, the TVECA is the tyrosine protein kinase receptor, Tie2. Tie2 is an endothelial-specific receptor tyrosine kinase that has been shown to be overexpressed in the neovascular endothelium of human hepatocellular carcinoma (Tanaka et al., Hepatology 35:861-7, 2002). Tie2 is a type I membrane protein consisting of 1124 amino acids. Sequence analysis indicates that this protein consists of a signal peptide, an extracellular domain (containing 3 EGF-like domains, 3 fibronectin type-III domains, and 2 Ig-like C2-type domains), a transmembrane domain, and a cytoplasmic domain.

An exemplary amino acid sequence of human Tie2 can be found in Swiss-Prot Accession No. Q02763. This protein consists of 1124 amino acids and comprises the following domains: a signal peptide (amino acid residues 1-22), an extracellular domain (amino acid residues 23-745), a transmembrane domain (amino acid residues 746-770), and a cytoplasmic domain (amino acid residues 771-1124). In some embodiments the TVECA may be native, recombinant, or synthetic Tie2. In other embodiments, the TVECA may be a Tie2 protein or fragment or a nucleic acid encoding a Tie2 or a Tie2 fragment. Preferred fragments of Tie2 include all or a portion of the extracellular domain. The invention methods can use variants or splice variants of aTie2 or a Tie2 having post-translational modifications.

The term “mucin” as used herein refers to any of a class of high molecular weight glycoproteins with a high content of clustered oligosaccharides O-glycosidically linked to tandem repeating peptide sequences which are rich in threonine, serine and proline. Mucin plays a role in cellular protection and, with many sugars exposed on the extended structure, effects multiple interactions with various cell types including leukocytes and infectious agents. Mucin antigens also include those identified as CD227, Tumor-associated epithelial membrane antigen (EMA), Polymorphic epithelial mucin (PEM), Peanut—reactive urinary mucin (PUM), episialin, Breast carcinoma-associated antigen DF3, H23 antigen, mucin 1, Episialin, Tumor-associated mucin, Carcinoma-associated mucin. Also included are CA15-3 antigen, M344 antigen, Sialosyl Lewis Antigen (SLA), CA19-9, CA195 and other mucin antigen previously identified by monoclonal antibodies (e.g., see U.S. Pat. No. 5,849,876). The term mucin does not include proteoglycans which are glycoproteins characterized by glycosaminoglycan chains covalently attached to the protein backbone.

At least 15 different mucins have been described including MUC1, MUC2, MUC3A, MUC3B, MUC4, MUC5AC, MUC5B, MUC6, MUC7, MUC8, MUC9, MUC12, MUC13, MUC15, and MUC16 (these may also be designated with a hyphen between “MUC” and the number). The nucleotide sequence and amino acid sequence of these mucins are known. NCBI and Swiss Prot accession nos. for exemplary sequences of each of these mucins are as follows: MUC1 (NCBI NM_{—}002456, Swiss Prot P15941), MUC2, (NCBI NM_{—}002457, Swiss Prot Q02817) MUC3A (NCBI AF113616, Swiss Prot Q02505), MUC3B (NCBI AJ291390, Swiss Prot Q9H195), MUC4 (NCBI NM_{—}138299, Swiss Prot Q99102), MUC5AC (NCBI AF043909, Swiss Prot Q8WWQ5), MUC5B (Swiss Prot Q9HC84), MUC6 (NCBI U97698, Swiss Prot Q8N8I1), MUC7 (NCBI L42983, Swiss Prot Q8TAX7), MUC8 (NCBI U14383, Swiss Prot Q12964), MUC9 (NCBI U09550, Swiss Prot Q12889), MUC12 (Swiss Prot Q9UKN1), MUC13 (NCBI NM_{—}017648, Swiss Prot Q9H3R2), MUC15 (NCBI NM_{—}145650, Swiss Prot Q8WW41), and MUC16 (NCBI AF361486, Swiss Prot Q8WXI7; aka CA125).

There are two structurally and functionally distinct classes of mucins: secreted gel-forming mucins (MUC2, MUC5AC, MUC5B, and MUC6) and transmembrane mucins (MUC1, MUC3A, MUC3B, MUC4, MUC12, MUC17). The products of some MUC genes do not fit well into either class (MUC7, MUC8, MUC9, MUC13, MUC15, MUC16).

The characteristics of particular mucins as TAA in particular cancers is supported by alterations in expression and structure in association with pre-neoplastic and neoplastic lesions (Filipe M I: Invest Cell Pathol 1979, 2:195-216; Filipe M I, Acta Med Port 1979, 1:351-365). For instance, normal mucosa of the stomach is characterized by the expression of MUC1, MUC5A/C, MUC6 mRNA and the encoded immunoreactive protein. Also, high levels of MUC2, MUC3 mucin mRNA and encoded immunoreactive protein are associated with intestinal metaplasia. Gastric cancer exhibits markedly altered secretory mucin mRNA levels compared with adjacent normal mucosa, with decreased levels of MUC5 and MUC6 mRNA and increased levels of MUC3 and MUC4 mRNA. High levels of MUC2 and MUC3 mRNA and protein are detectable in the small intestine, and MUC2 is the most abundant colonic mucin.

Mucins represent diagnostic markers for early detection of pancreatic cancer and other cell types. Studies have shown, that ductal adenocarcinomas (DACs) and tumor cell lines commonly overexpress MUC1 mucin. See Andrianifahanana et al., Clin Cancer Res 2001, 7:4033-4040. This mucin was detected only at low levels in the most chronic pancreatitis and normal pancreas tissues but is overexpressed in all stages of pancreatic cancers. The de novo expression of MUC4 in pancreatic adenocarcinoma and cell lines has been reported (Hollingsworth et al., Int J Cancer 1994, 57:198-203). MUC4 mRNA expression has been observed in the majority of pancreatic adenocarcinoma and established pancreatic cancer cell lines but not in normal pancreas or chronic pancreatitis tissues. MUC 4 expression also has been associated with lung cancer (see Nguyen et al. 1996 Tumor Biol. 17:176-192). MUC5 is associated with metastases in non-small cell lung cancer (see Yu et al., 1996 Int. J. Cancer 69:457-465). MUC6 is overexpressed and MUC5AC is de novo expressed in gastric and invasive DACs (Kim et al., Gastroenterology 2002, 123:1052-1060). MUC7 has been reported as a marker for invasive bladder cancer (see Retz et al. 1998 Cancer Res. 58:5662-5666).

Expression of the MUC2 secreted gel-forming mucin is generally decreased in colorectal adenocarcinoma, but preserved in mucinous carcinomas, a distinct subtype of colon cancer associated with microsatellite instability. MUC2 is increased in laryngeal cancer (Jeannon et al. 2001 Otolaryngol Head Neck Surg. 124:199-202). Another secreted gel-forming mucin, MUC5AC, a product of normal gastric mucosa, is absent from normal colon, but frequently present in colorectal adenomas and colon cancers.

MUC1, also known as episialin, polymorphic epithelial mucin (PEM), mucin like cancer associated antigen (MCA), CA27.29, peanut-reactive urinary mucin (PUM), tumor-associated epithelial mucin, epithelial membrane antigen (EMA), human milk fat globule (HMFG) antigen, MUC1/REP, MUC1/SEC, MUC1Y, CD227, is the most well known of the mucins. The gene encoding MUC1 maps to 1q21-q24. The MUC1 gene contains seven exons and produces several different alternatively spliced variants. The tandem repeat domain is highly O-glycosylated and alterations in glycosylation have been shown in epithelial cancer cells.

MUC1 mRNA is polymorphic in size. There are presently nine isoforms of MUC1 (Swiss-Prot Accession No. P15941) based on alternate splicing (isoform: isoform ID; 1: ID P15941-1, 2: ID P15941-2, 3: ID P15941-3, 4: ID P15941-4, 5: P15941-5, 6: ID P15941-6, 7: ID P15941-7, 8: ID P15941-8, and 9: ID P15941-9).

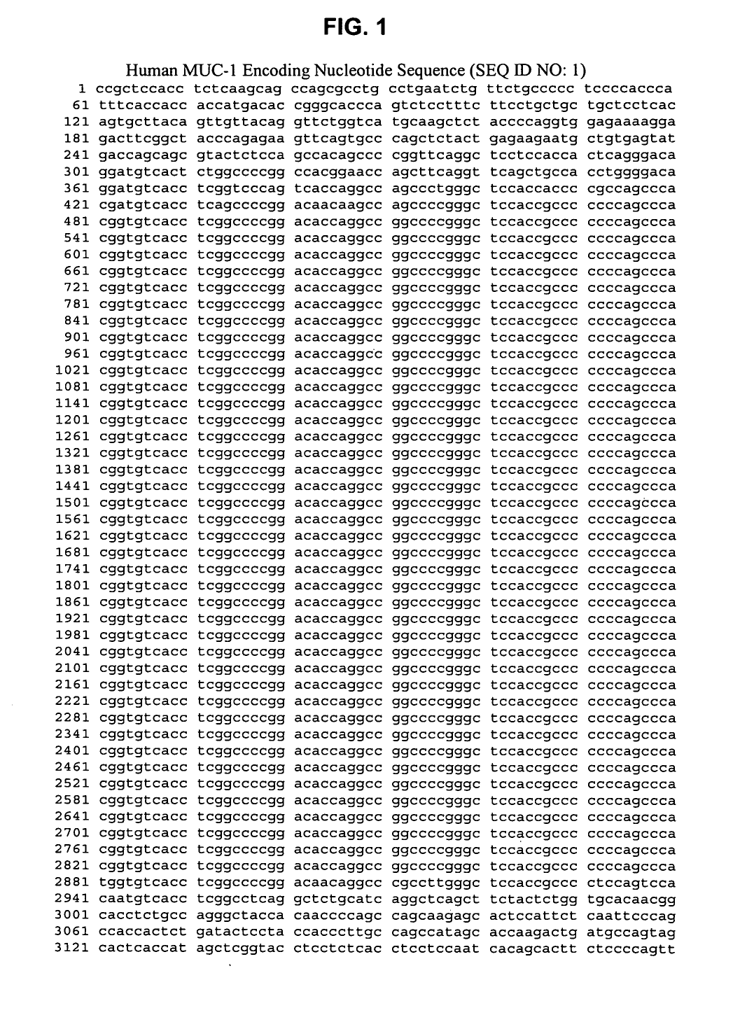

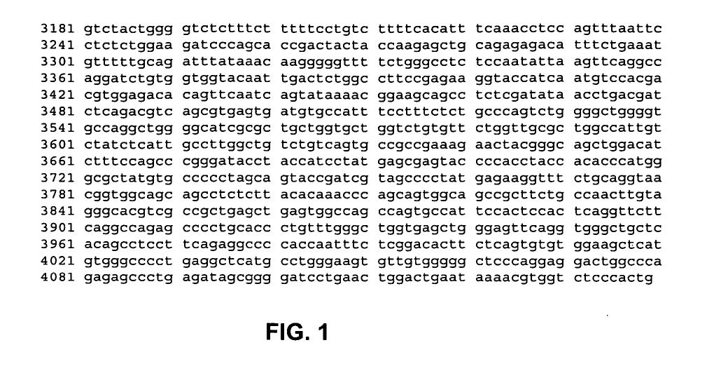

MUC1 isoform 1 (aka. MUC1/REP) is a polymorphic, type I transmembrane protein containing: 1) a large extracellular domain, primarily consisting of a 20-amino acid (aa) repeat motif (a region known as Variable Number (30-100) of tandem repeats—VNTR); 2) a transmembrane domain; and 3) a 72-aa cytoplasmic tail. During biosynthesis, the MUC1/REP protein is modified to a large extent, and a considerable number of O-linked sugar moieties confer mucin-like characteristics on the mature protein. Soon after translation, MUC1/REP is cleaved into two products that form a tightly associated heterodimer complex composed of a large extracellular domain, linked noncovalently to a much smaller protein including the cytoplasmic and transmembrane domains. The extracellular domain can be shed from the cell. Using Swiss Prot P15941 as a reference (see FIG. 1), the extracellular domain (ecm) of MUC1 isoform 1 represents amino acids 24 to 1158, the transmembrane domain represents 1159-1181, and the cytoplasmic domain represents 1182-1255. The SEA domain represents is 1034-1151 and represents a C-terminal portion of what is referred to as the extracellular domain. The SEA domain of a mucin is generally a target for proteolytic cleavage, yielding two subunits, the smaller of which is associated with the cell membrane.

MUC1 isoform 5 (aka MUC1/SEC) is a form of MUC1 that is secreted by cells. It has an extracellular domain that is identical to that of isoform 1 (MUC1/REP), but lacks a transmembrane domain for anchoring the protein to a cell membrane. MUC1 isoform 7 (aka MUC1/Y) contains the cytoplasmic and transmembrane domains observed in isoforms 1 (MUC1/REP) and 5 (MUC1/SEC), but has an extracellular domain that is smaller than MUC, lacking the repeat motif and its flanking region (see Baruch A. et al., 1999 Cancer Res. 59, 1552-1561). Isoform 7 behaves as a receptor and binds the secreted isoform 5. Binding induces phosphorylation of isoform 7 and alters cellular morphology and initiates cell signaling through second messenger proteins such as GRB2, (see Zrihan-Licht S. et al., 1995 FEBS Lett. 356, 130-136). It has been shown that β-catenin interacts with the cytoplasmic domain of MUC1 (Yamamoto M. et al., 1997 J. Biol. Chem. 272, 12492-12494).

MUC1 is expressed focally at low levels on normal epithelial cell surfaces. See 15. Greenlee, et al., Cancer Statistics CA Cancer J. 50, 7-33 (2000); Ren, et al., J. Biol. Chem. 277, 17616-17622 (2002); Kontani, et al., Br. J. Cancer 84, 1258-1264 (2001); Rowse, et al., Cancer Res. 58, 315 (1998). MUC1 is overexpressed in carcinomas of the breast, ovary, pancreas as well as other carcinomas (see also Gendler S. J. et al, 1990 J. Biol. Chem. 265, 15286-15293). A correlation is found between acquisition of additional copies of MUC1 gene and high mRNA levels (p<0.0001), revealing the genetic mechanism responsible for MUC1 gene overexpression, and supporting the role of MUC1 gene dosage in the pathogenesis of breast cancer (Bièche I. et al.,. 1997 Cancer Genet. Cytogenet. 98, 75-80). MUC1 mucin, as detected immunologically, is increased in expression in colon cancers, which correlates with a worse prognosis and in ovarian cancers.

High level expression of the MUC1 antigen plays a role in neoplastic epithelial mucosal cell development by disrupting the regulation of anchorage dependent growth (disrupting E-cadherin function), which leads to metastases. See Greenlee, et al., Cancer Statistics CA Cancer J. 50, 7-33 (2000); Ren, et al. J. Biol. Chem. 277, 17616-17622 (2002). Non-MHC-restricted cytotoxic T cell responses to MUC1 have been reported in patients with breast cancer. See Kontani et al., Br. J. Cancer 84, 1258-1264 (2001). Human MUC1 transgenic mice (“MUC-1.Tg”) have been reported to be unresponsive to stimulation with human MUC1 antigen. See Rowse, et al., Cancer Res. 58, 315 (1998). Human MUC1 transgenic mice are useful for evaluating the development of immunity to MUC1 as a self antigen.

MUC1 protein and mRNA have been found in the ER-positive MCF-7 and BT-474 cells as well as in the ER-negative MDA-MB-231 and SK-BR-3 BCC cells. The mRNA Transcript level was higher in ER+ than in ER− cell lines. MUC1 reacts with intracellular adhesion molecule-1 (ICAM-1). At least six tandem repeats of MUC1 are needed (Regimbald et al., 1996 Cancer Res. 56, 4244-4249). The tandem repeat peptide of MUC1 from T-47D BCC was found to be highly O-glycosylated with 4.8 glycosylated sites per repeat, which compares to 2.6 sites per repeat for the mucin from milk.

The term “mucin antigen” as used herein refers to the full length mucin or a portion of a mucin that contains an epitope characterized in being able to elicit cellular immunity using a MUC-CD40L expression vector administered in vivo as described herein. A “mucin antigen” includes one or more epitopes from the extracellular domain of a mucin such as one or more of the tandem repeat motifs associated with the VNTR, or the SEA region. A mucin antigen may contain the entire extracellular domain. Also included within the meaning of “mucin antigen” are variations in the sequence including conservative amino acid changes and the like which do not alter the ability of the antigen to elicit an immune response that crossreacts with a native mucin sequence.

The VNTR consists of variable numbers of a tandemly repeated peptide sequences which differ in length (and composition) according to a genetic polymorphism and the nature of the mucin. The VNTR may also include 5′ and 3′ regions which contain degenerate tandem repeats. For example, in MUC1, the number of repeats varies from 21 to 125 in the northern European population. In the U.S. the most infrequent alleles contains 41 and 85 repeats, while more common alleles have 60-84 repeats. The MUC1 repeat has the general repeating peptide sequence PDTRPAPGSTAPPAHGVTSA (SEQ ID NO:12). Underlying the MUC1 tandem repeat is a genetic sequence polymorphism at three positions shown bolded and underlined (positions 2, 3 and 13). The concerted replacement DT→ES (sequence variation 1) and the single replacements P→Q (sequence variation 2), P→A (sequence variation 3), and P→T (sequence variation 4) have been identified and vary with position in the domain (see Engelmann et al., 2001 J. Biol. Chem. 276:27764-27769). The most frequent replacement DT→ES occurs in up to 50% of the repeats. Table 1 shows some exemplary tandem repeat sequences.

TABLE 1
Mucin Tandem Repeat Sequences

| | Tandem Repeat | |
|---|---|---|
| Mucin | (SEQ ID NO:) | Mucin source |
| MUC1 | PDTRPAPGSTAPPAHGVTSA | Mammary |
| | (SEQ ID NO:12) | |
| | PDNKPAPGSTAPPAHGVTSA | Pancreatic |
| | (SEQ ID NO:13) | |
| MUC2 | PTTTPPITTTTTVTPTPTPTGTQT | Intestinal |
| | (SEQ ID NO:14) | Tracheobronchial |
| MUC3 | HSTPSFTSSITTTETTS | Intestinal |
| | (SEQ ID NO:15) | Gall Bladder |
| MUC4 | TSSASTGHATPLPVTD | Colon |
| | (SEQ ID NO:16) | Tracheobronchial |
| MUC5AC | TTSTTSAP | Gastric |
| | (SEQ ID NO:17) | Tracheobronchial |
| MUC5B | SSTPGTAHTLTMLTTTATTPTATGSTA | Tracheobronchial |
| | TP | Salivary |
| | (SEQ ID NO:18) | |
| MUC7 | TTAAPPTPSATTPAPPSSSAPG | Salivary |
| | (SEQ ID NO:19) | |
| MUC8 | TSCPRPLQEGTPGSRAAHALSRRGHRV | Tracheobronchial |
| | HELPTSSPGGDTGF | |
| | (SEQ ID NO:20) | |

Although a mucin antigen as used herein may comprise only a single tandem repeat sequence motif, it should be understood that the immune response will generally be stronger and more efficiently generated if the vector encodes multiple such repeats. The invention vector preferably encodes mucin tandem repeats from 2-4, more preferably from 5-9, even more preferably from 10-19, yet even more preferably from 20-29, still more preferably from 30-39, and still yet more preferably from 40-50. Tandem repeats greater than 50 are possible and may include the number of such repeats found in natural mucins.

A mucin antigen as this term is used herein also may encompass tandem repeats from different types of mucins. For example, an expression vector may encode tandem repeats from two different mucins, e.g., MUC1 and MUC2. Such a vector also may encode multiple forms of the SEA domain as well or a combination of tandem repeats and one or more SEA domains.

A secretable form of an antigen is one that lacks all or substantially all of its transmembrane domain, if present in the mature protein. For example, in the case of a TVECA, the transmembrane domain, if present, is generally about 20-30 amino acids in length and functions to anchor TVECA or a fragment thereof in the cell membrane. A TVECA missing substantially all of the transmembrane is one where the domain comprises 6 residues or less, more preferably less than about 4 residues of sequence, even more preferably less than about 2 residues of sequence and most preferably 1 residue or less of sequence. Any transmembrane sequence that is present may be at one end of the domain or may be divided between both ends. In a preferred embodiment, the vaccine vector transcription unit encodes a secretable form of a TVECA lacking the entire transmembrane domain. The extracellular domain of a human TVECA is denoted herein as “ecdTVECA.” For example, the extracellular domain of VEGF R1 is denoted “ecdVEGFR1.”

It should be understood that a TVECA which lacks a functional transmembrane domain may still include all or a portion of the cytoplasmic domain or any other domain (excluding the transmembrane domain).

DNA encoding the various annexin A1, annexin A8, VEGF R1, endosialin, and Tie2 antigens may be obtained from the RNA of cell lines expressing the antigen, using a commercial cDNA synthesis kit and amplification using a suitable pair of PCR primers that can be designed from the published DNA sequences. Annexin A1, annexin A8, VEGF R1, endosialin, or Tie2 encoding DNA also may be obtained by amplification from RNA or cDNA obtained or prepared from human or other animal tissues. For DNA segments that are not that large, the DNA may be synthesized using an automated oligonucleotide synthesizer.

The terms “linker” and “spacer” are used interchangeably. These terms as used herein with respect to the transcription unit of the expression vector, refer to one or more amino acid residues between the carboxy terminal end of the antigen and the amino terminal end of CD40 ligand. The composition and length of the linker may be determined in accordance with methods well known in the art and may be tested for efficacy. See e.g. Arai et al., design of the linkers which effectively separate domains of a bifunctional fusion protein. Protein Engineering, Vol. 14, No. 8, 529-532, August 2001. The linker is generally from about 3 to about 15 amino acids long, more preferably about 5 to about 10 amino acids long, however, longer or shorter linkers may be used or the linker may be dispensed with entirely. Longer linkers may be up to about 50 amino acids, or up to about 100 amino acids. A short linker 10 residues or less is preferred when the mucin antigen is N-terminal to the CD40 ligand. One example of a linker well-known in the art is a 15 amino acid linker consisting of three repeats of four glycines and a serine (i.e., [Gly₄Ser]₃).

The term “CD40 ligand” (CD40L) as used herein refers to a full length or portion of the molecule known also as CD154 or TNF5. CD40L is a type II membrane polypeptide having a cytoplasmic domain at its N-terminus, a transmembrane region and then an extracellular domain at its C-terminus. Unless otherwise indicated the full length CD40L is designated herein as “CD40L,” “wtCD40L” or “wtTmCD40L.” The form of CD40L in which the cytoplasmic domain has been deleted is designated herein as “ΔCtCD40L.” The form of CD40L where the transmembrane domain has been deleted is designated herein as “ΔTmCD40L.” The form of CD40L where both the cytoplasmic and transmembrane domains have been deleted is designated herein as “ΔCtΔTmCD40L” or “ecdCD40L.” The nucleotide and amino acid sequences of CD40L from mouse and human are well known in the art and can be found, for example, in U.S. Pat. No. 5,962,406 (Armitage et al.). Also included within the meaning of CD40 ligand are variations in the sequence including conservative amino acid changes and the like which do not alter the ability of the ligand to elicit an immune response to a TVECA in the fusion protein of the invention.

Murine CD40L (mCD40L) is 260 amino acids in length. The cytoplasmic (Ct) domain of mCD40L extends approximately from position 1-22, the transmembrane domain extends approximately from position 23-46, while the extracellular domain extends approximately from position 47-260.

Human CD40L (hCD40L) is 261 amino acids in length. The cytoplasmic domain of hCD40L extends approximately from position 1-22, the transmembrane domain extends approximately from position 23-46, while the extracellular domain extends approximately from position 47-261.

The phrase “CD40 ligand is missing all or substantially all of the transmembrane domain rendering CD40 ligand secretable” as used herein refers to a recombinant form of CD40 ligand that can be secreted from a cell. The transmembrane domain of CD40L which contains about 24 amino acids in length, functions to anchor CD40 ligand in the cell membrane. CD40L from which all of the transmembrane domain has been deleted is CD40 ligand lacking residues 23-46. CD40 ligand missing substantially all of the transmembrane is one that comprises 6 residues or less, more preferably less than about 4 residues of sequence, even more preferably less than about 2 residues of sequence and most preferably 1 residue or less of sequence. Any transmembrane sequence that is present from the CD40L may be at one end of the domain or may be divided between both ends. In a preferred embodiment, the vaccine vector transcription unit encodes a secretable form of a TVECA wherein CD40L is lacking the entire transmembrane domain.

The extracellular domain of a human TVECA is denoted herein as “ecdTVECA.” For example, the extracellular domain of VEGF R1 is denoted “ecdVEGFR1.”

6 residues or less of sequence at one end of the transmembrane domain, more preferably less than about 4 residues of sequence at one end of the transmembrane domain, even more preferably less than about 2 residues of sequence on one end of the transmembrane domain, and most preferably 1 residue or less on one end of the transmembrane domain. Thus, a CD40L that lacks substantially all of the transmembrane domain rendering the CD40L secretable is one that retains no more than six residues of sequence on one end of the domain. Such as CD40L would contain, in addition to the extracellular domain and optionally the cytoplasmic domain, and no more than amino acids 41-46 or 23-28 located in the transmembrane domain of CD40L. In a preferred embodiment, the vaccine vector transcription unit encodes a secretable form of CD40 containing less than 10% of the transmembrane domain. More preferably, CD40L contains no transmembrane domain.

It should be understood that a CD40L which lacks a functional transmembrane domain may still include all or a portion of the cytoplasmic domain. Likewise, a CD40L which lacks a functional transmembrane domain may include all or a substantial portion of the extracellular domain.

As used herein, an expression vector and fusion protein boost is administered as a vaccine to achieve cancer immunotherapy. The expression vector and protein boost may be formulated as appropriate with a suitable pharmaceutically acceptable carrier. Accordingly, the vectors or protein boost may be used in the manufacture of a medicament or pharmaceutical composition. Expression vectors and the fusion protein may be formulated as solutions or lyophilized powders for parenteral administration. Powders may be reconstituted by addition of a suitable diluent or other pharmaceutically acceptable carrier prior to use. Liquid formulations may be buffered, isotonic, aqueous solutions. Powders also may be sprayed in dry form. Examples of suitable diluents are normal isotonic saline solution, standard 5% dextrose in water, or buffered sodium or ammonium acetate solution. Such formulations are especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride, sodium citrate, and the like.

Alternately, expression vectors and the fusion protein may be prepared for oral administration. Pharmaceutically acceptable solid or liquid carriers may be added to enhance or stabilize the composition, or to facilitate preparation of the vectors. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. When a liquid carrier is used, the preparation may be in the form of a syrup, elixir, emulsion, or an aqueous or non-aqueous suspension.

Expression vectors and the fusion protein may be formulated to include other medically useful drugs or biological agents. The vectors also may be administered in conjunction with the administration of other drugs or biological agents useful for the disease or condition that the invention compounds are directed.

As employed herein, the phrase “an effective amount,” generally refers to a dose sufficient to provide concentrations high enough to generate (or contribute to the generation of) an immune response in the recipient thereof. The specific effective dose level for any particular subject will depend upon a variety of factors including the disorder being treated, the severity of the disorder, the activity of the specific compound, the route of administration, the rate of clearance of the viral vectors, the duration of treatment, the drugs used in combination or coincident with the viral vectors, the age, body weight, sex, diet, and general health of the subject, and like factors well known in the medical arts and sciences. Various general considerations taken into account in determining the “therapeutically effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th ed., Mack Publishing Co., Easton, Pa., 1990. For administration of vectors, the range of particles per administration typically if from about 1×10⁷ to 1×10¹¹, more preferably 1×10⁸ to 5×10¹⁰, and even more preferably 5×10⁸ to 2×10¹⁰. A vector can be administered parenterally, such as intravascularly, intravenously, intraarterially, intramuscularly, subcutaneously, or the like. Administration can also be orally, nasally, rectally, transdermally or inhalationally via an aerosol. The vectors may be administered as a bolus, or slowly infused. The vector is preferably administered subcutaneously.

As demonstrated herein, vectors encoding tumor antigens can induce a protective cellular and humoral immunity against such antigens, including those to which tolerance had developed. Although not wishing to be bound by any theory, it is believed that the vaccines provided herein generate upon administration a continual local release of the fusion protein composed of the secretable form of the antigen linked to a secretory form of CD40 ligand. As demonstrated herein this facilitates DCs maturation, promoting the development of effective antigen-specific immunity. In particular, it is demonstrated herein that the fusion protein produced by an adenoviral vector encoding a secretable fusion protein comprising human AnxA1 and the murine CD40L lacking a transmembrane and cytoplasmic domain (i.e. Ad-sig-AnxA1/ecdCD40L), induced regression of the hMUC-1 positive cells in hMUC-1.Tg mice. Although not wishing to be bound by any theory, it is believed that subcutaneous injection of the Ad-sig-AnxA1/ecdCD40L vector elicited strong AnxA1-specific CD4⁺ and CD8⁺ T cell-mediated immunity.

The immunity generated against the antigens using the invention methods is long lasting. As used herein, the term long lasting means that immunity elicited by the antigen encoded by the vector can be demonstrated for up to 6 months from the last administration, more preferably for up to 8 months, more preferably for up to one year, more preferably up to 1.5 years, and more preferably for at least two years.

In one embodiment, immunity to a TVECA can be generated by producing a fusion protein that comprises the extracellular domain of a TVECA to the amino-terminal end of the CD40 ligand from which the transmembrane and cytoplasmic domains were deleted. In one example, the TVECA is annexin A1.

Although not wishing to be bound by any theory, it is believed that the cells infected in the vicinity of the site of subcutaneous injection of the vector release the antigen/CD40 ligand secretory which is taken up by antigen presenting cells (e.g. DCs) in the vicinity of the infected cells. The internalized antigen would be digested in the proteosome with the resultant antigen peptides trafficking to the endoplasmic reticulum where they would bind to Class I MHC molecules. Eventually, the DCs would present the antigen on the surface in the Class I MHC molecule. Activated, antigen-loaded antigen presenting cells would migrate to lymphocyte bearing secondary organs such as the regional lymph nodes or the spleen. During the two weeks of continuous release of the tumor antigen/CD40 fusion protein, CD8 cytotoxic T cell lymphocytes competent to recognize and kill cells, which carried the antigens, would be expanded in the lymph nodes and spleen by the presence of the activated and antigen loaded dendritic cells. The continuous nature of the stimulation and the expansion of the tumor antigen specific cytotoxic T cells by the continuous release from the vector infected cells is believed to generate an immune response which would be greater in magnitude than is possible using a vector which carried a tumor antigen/CD40 ligand which is non-secretory.

The methods of the present invention, therefore, can be used to generate immunity to an antigen which is a self-antigen in an individual. For example, a vector that encodes an annexin A1 antigen can be used to generate CD8⁺ immunity in a human where the annexin A1 antigen is a self antigen. The invention methods also can be used to overcome a state of immunological anergy to an antigen which is a self-antigen.

The following examples serve to illustrate the present invention. These examples are in no way intended to limit the scope of the invention.

### EXAMPLES

1. Construction of Adenoviral Expression Vectors

The transcription unit, sig-ecdhMUC1-ΔCtΔTmCD40L of the adenoviral vector encodes a signal sequence (from an Ig kappa chain) followed by the extracellular domain of human MUC1 which is connected via a linker to a fragment of the CD40 ligand (human or mouse) which contains the extracellular domain without the transmembrane or cytoplasmic domains. The fusion protein was engineered to be secreted from vector infected cells by the addition of the kappa chain signal sequence to the amino-terminal end of the fusion protein.

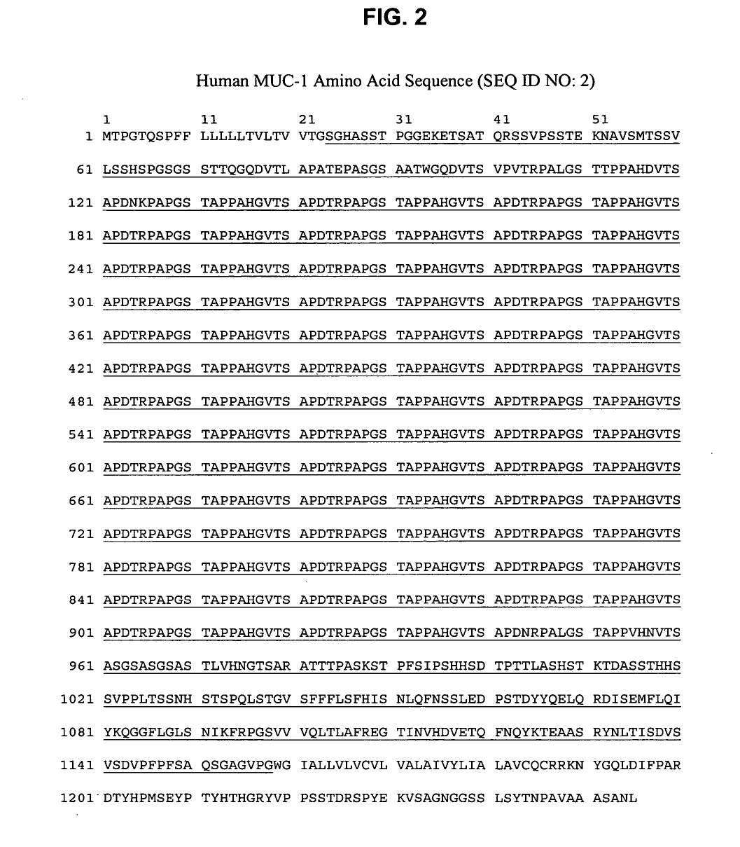

The amino acid sequence of human MUC-1 and the encoding nucleotide sequence are shown in FIGS. 2 and 1, respectively. The encoded MUC1 protein represents 1255 amino acids encoded by nucleotides 74 to 3,841 of SEQ ID NO: 1. The first 23 amino acids (encoded by nucleotides 74 to 142 of SEQ ID NO:1) represent the MUC1 signal sequence which is removed from the mature mucin. The extracellular domain represents about 1135 amino acids from positions 24 to 1158 (encoded by nucleotides 143 to 3547). The tandem repeat region represents approximately 900 amino acids. Amino acids 74 to 126 (encoded by 296 to 451 of SEQ ID NO:1) represents a 5′ degenerate tandem repeat region, amino acids 127 to 945 represents the tandem repeat region (encoded by 452 to 2,908 of SEQ ID NO: 1) while amino acids 946 to 962 represent a 3′ degenerate tandem repeat region (encoded by 2909 to 2959 of SEQ ID NO: 1). The SEA domain represents amino acids 1034 to 1151, the transmembrane domain represents amino acids 1159 to 1181, and the cytoplasmic domain represents amino acids 1182 to 1255 (see SEQ ID NO:2).

The transcription unit was introduced into the E1 gene region of the adenoviral vector backbone. After the adenoviral vector particles were generated in HEK 293 cells, the vector DNA was purified by cesium chloride gradient centrifugation. The presence of the signal peptide in the adenoviral vector was confirmed by restriction enzyme analysis and by DNA sequencing.

A transcription unit that included DNA encoding the signal sequence of the mouse IgG kappa chain gene upstream of DNA encoding amino acid residues 95-159 of human MUC-1 (“sig-ecdhMUC-1”) was generated by PCR using plasmid pcDNA3-hMUC-1 (gift of Finn O. J., University of Pittsburgh School of Medicine) and the primers below. DNA encoding the mouse IgG kappa chain METDTLLLWVLLLWVPGSTGD (single letter amino acid code) (SEQ ID NO:21) was prepared by PCR amplification (using the primers set forth in SEQ ID NOs:22, 23, and 24), in conjunction with the amplification of hMUC-1 (using the primers set forth in SEQ ID NOs:25 and 26) to generate the full 21 amino acid mouse IgG kappa chain signal sequence (the start codon “ATG” is shown bolded in SEQ ID NO:22) upstream of the hMUC-1 antigen.

| | |
|---|---|
| Primer 1: | |
| 5′-CCACC ATG GAG ACA GAC ACA CTC CTG | (SEQ ID NO:22) |
| CTA TGG GTA CTG CTG-3′; | |
| primer 2: | |
| 5′- TC CTG CTA TGG GTA CTG CTG CTC | (SEQ ID NO:23) |
| TGG GTT CCA GGT TC-3′; | |
| primer 3: | |
| 5′- TG CTC TGG GTT CCA GGT TCC ACT | (SEQ ID NO:24) |
| GGT GAC GAT G-3′; | |
| primer 4: | |
| 5′- GGT TCC ACT GGT GAC GAT GTC ACC | (SEQ ID NO:25) |
| TCG GTC CCA GTC-3′ | |
| (forward primer for MUC-1 repeat | |
| region); | |
| and | |
| primer 5: | |
| 5′- GAGCTCGAG ATT GTG GAC TGG AGG | (SEQ ID NO:26) |
| GGC GGT G-3′ | |
| (reverse primer for MUC-1 repeat | |
| region, Xho I cloning site in bold | |
| and underlined). | |

sig-ecdhMUC-1 with the upstream kappa signal sequence was generated by four rounds of PCR amplification (1^{st} round: primers SEQ ID NOs: 25 and 26; 2^{nd} round: primer SEQ ID NOs: 24 and 26; 3^{rd} round: primer SEQ ID NOs: 23 and 26; 4^{th} round: primer SEQ ID NOs: 22 and 26), under the following conditions: hold 3 min at 94° C.; cycle 94° C. for 50 sec, 52° C. for 50 sec, 72° C. for 30 sec (35 cycles); hold 7 min at 72° C.; and hold at 4° C. The sig-ecdhMUC-1 encoding DNA amplicon was cloned into the pcDNA™ 3.1 TOPO vector (Invitrogen, San Diego, Calif.) forming pcDNA-sig-ecdhMUC-1.

pShuttle-ΔCtΔTmCD40L (no signal sequence and the extracellular domain of murine CD40L) was prepared as follows. Plasmid pDC406-mCD40L was purchased from the American Type Culture Collection. A pair of PCR primers (SEQ ID NOs:27 and 28) was designed to amplify the mouse CD40 ligand from position 52 to 260 (i.e., CD40 ligand without the cytoplasmic and transmembrane domains). The forward primer includes sequence encoding a linker (indicated as “+spacer”) at the 5′ end of the amplicon.

Mouse ΔCtΔTmCD40L+spacer forward primer (MCD40LSPF) (CD40L sequence italicized; Xho I cloning site in bold and underlined):

| | |
|---|---|
| 5′- CCGCTCGAG AAC GAC GCA CAA GCA | (SEQ ID NO:27) |
| CCA AAA TCA AAG GTC GAA GAG GAA GTA | |
| AAC-3′; | |
| and | |
| mouse CD40L reverse primer (MCD40LR; | |
| Xba I cloning site in bold and | |
| underlined) | |
| 5′-GCGGGCC CGCGGCCGCCGCTAG TCTAGA | (SEQ ID NO:28) |
| GAG TTT GAG TAA GCC AAA AGA TGA | |
| G-3′. | |

The forward primer MCD40LSPF encodes a 10 residue spacer (LENDAQAPKS; single letter code; SEQ ID NO:29) to be located between the mucin and the CD40 ligand (CD40L) of the transcription unit. PCR performed using the forward and reverse primers (SEQ ID NOs:27 and 28) and plasmid pDC406-mCD40L as the template resulted in PCR fragment “spacer+ΔCtΔTmCD40L,” which was inserted into the plasmid pcDNA-sig-ecdhMUC1 after restriction endonuclease digestion with XbaI (TCTAGA) and Xho I (CTCGAG). This vector was designated pcDNA-sig-ecdhMUC1/ΔCtΔTmCD40L.

A vector was produced that was otherwise the same except that it encoded full length CD40L rather than the truncated form. This vector was made using a CD40 forward primer that annealed to the start codon of murine CD40L. This vector is designated pShuttleCD40L (no signal sequence).

The sig-ecdhMUC1/ΔCtΔTmCD40L encoding DNA was cut from the pcDNA3TOPO vector using HindIII-XbaI restriction and inserted into pShuttle-CMV (see Murphy et al., Prostate 38: 73-78, 1999) downstream of the CMV promoter. The plasmid is designated pShuttle-sig-ecdhMUC1-ΔCtΔTmCD40L. Thus, the transcription unit sig-ecdhMUC1-ΔCtΔTmCD40L encodes the mouse IgG kappa chain secretory signal followed by the extracellular domain of human MUC1 followed by a 10 amino acid linker with (LENDAQAPKS; SEQ ID NO:29) followed by murine CD40 ligand residues 52-260.

A transcription unit (SEQ ID NO:30, below) that included DNA encoding the signal sequence of the human IL-2 gene (italics) upstream of DNA encoding amino acid residues 95-159 of human MUC-1 (boxed sequence) and connected upstream of human CD40 ligand (underlined) via a spacer sequence (shaded sequence) was constructed as follows.

ATGTATAGGATGCAACTGCTGTCTTGCATTGCTCTGTCTCTGGCACTGGTCACTA
ACTCTGCCAGGATCCACCTCTGGGTTCCAGGTTCCACTGGTGAC

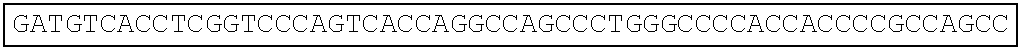

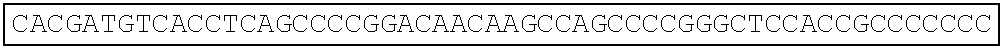

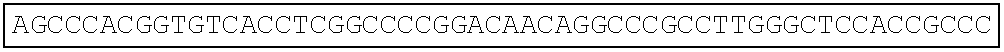

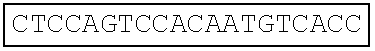

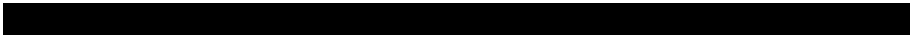

GTGTATCTTCATAGAAGGTTGGACAAGATAGAAGATGAAAGGAATCTTCATGAAGA
TTTTGTATTCATGAAAACGATACAGAGATGCAACACAGGAGAAAGATCCTTATCCTT
ACTGAACTGTGAGGAGATTAAAAGCCAGTTTGAAGGCTTTGTGAAGGATATAATGTT
AAACAAAGAGGAGACGAAGAAAGAAAACAGCTTTGAAATGCAAAAAGGTGATCAG
AATCCTCAAATTGCGGCACATGTCATAAGTGAGGCCAGCAGTAAAACAACATCTGT
GTTACAGTGGGCTGAAAAAGGATACTACACCATGAGCAACAACTTGGTAACCCTGG
AAAATGGGAAACAGCTGACCGTTAAAAGACAAGGACTCTATTATATCTATGCCCAA
GTCACCTTCTGTTCCAATCGGGAAGCTTCGAGTCAAGCTCCATTTATAGCCAGCCTCT
GCCTAAAGTCCCCCGGTAGATTCGAGAGAATCTTACTCAGAGCTGCAAATACCCAC
AGTTCCGCCAAACCTTGCGGGCAACAATCCATTCACTTGGGAGGAGTATTTGAATTG
CAACCAGGTGCTTCGGTGTTTGTCAATGTGACTGATCCAAGCCAAGTGAGCCATGGC
ACTGGCTTCACGTCCTTTGGCTTACTCAAACTCTGA-3′ (SEQ ID NO: 30).

A nucleic acid fragment encoding amino acid residues 95-159 of human MUC-1 was amplified from pcDNA-sig-ecdhMUC-1/ΔCtΔTmCD40L by PCR using the following below (SEQ ID NOs:31 and 32) under the following conditions: hold 3 min at 94° C.; cycle 94° C. for 50 sec, 52° C. for 50 sec, 72° C. for 30 sec (35 cycles); hold 7 min at 72° C.; and hold at 4° C.

| | |
|---|---|
| forward primer | |
| (Bam HI site in bold and | |
| underlined): | |
| 5′- CG GGATCC AC CTCTGGGTTCCAGGTTCCA | (SEQ ID NO:31) |
| CTGGTGAC-3′; | |
| and | |
| reverse primer | |
| (Eco RV site in bold and | |
| underlined): | |
| 5′-GCCGC GATATC TGC TTG TGC GTC GTT | (SEQ ID NO:32) |
| CTC GAG GG-3′. | |

The amplified fragment was inserted into the vector pVAC1-mcs (Invivogen Inc.), which contains the coding sequence of human IL-2 signal peptide upstream of the multiple cloning site, using the restriction enzyme sites BamHI and EcoRV. The resulting vector was named pVAC1-sig-ecdhMUC-1.

Human CD40L ligand was amplified with the primers below (SEQ ID NOs:33 and 34) and pcDNA-hCD40L as the template under the following conditions: hold 3 min at 94° C.; cycle 94° C. for 45 sec, 52° C. for 45 sec, 72° C. for 70 sec (30 cycles); hold 7 min at 72° C.; and hold at 4° c.

| | |
|---|---|
| Human CD40L forward primer | |
| (Eco RV site in bold and | |
| underlined): | |
| 5′- GC GATATC AAC GAC GCA CAA GCA | (SEQ ID NO:33) |
| CCA AAA TCA GTG-3′ | |
| and | |
| human CD40L reverse primer | |
| (Eco RI site in bold and | |
| underlined): | |
| 5′-CGG GAATTC TGC TCTAGA TCAGAGTTTGA | (SEQ ID NO:34) |
| G TAAGCCAAAG GAC-3′. | |

The amplified fragment was digested with Eco RV and Eco RI and the digested fragment inserted into pVAC1-sig-ecdhMUC-1 at the same sites. The resulting vector was named pVAC1-sig-ecdhMUC-1/humanCD40L.

The fusion construct (i.e., hMUC-1/human CD40 ligand) was PCR amplified using the primers below (SEQ ID NOs:35 and 36) and pVAC1-sig-ecdhMUC-1/humanCD40L as template under the following conditions: hold 3 min at 94° C.; cycle 94° C. for 50 sec, 55° C. for 50 sec, 72° C. for 90 sec (30 cycles); hold 7 min at 72° C.; and hold at 4° C.

| | |
|---|---|
| Fusion construct forward primer: | |
| 5′-AAAGCCATCATGTATAGGATGCAACTGCTGTCT | (SEQ ID NO:35) |
| TGC-3′ | |
| and | |
| fusion construct reverse primer: | |
| 5′-CGGGAATTC TGC TCTAGA TCAGAGTTTGAG | (SEQ ID NO:36) |
| TAAGCCAAAG GAC-3′. | |

The resulting amplified fragment was subcloned into the pcDNA3TOPO vector. The fusion protein-encoding DNA was cut from the pcDNA3TOPO vector using Kpn I and Xba I restriction enzymes and inserted into pShuttle-CMV (see Murphy et al., Prostate 38: 73-78, 1999) downstream of the CMV promoter. The resulting plasmid is designated pShuttle-sig-ecdhMUC1-ΔCtΔTmCD40L.

In some vectors, the mouse HSF1 trimer domain was added between the ecdhMUC1 encoding DNA and ΔCtΔTm CD40L encoding by PCR using plasmid pcDNA-sig-ecdhMUC1/ΔCtΔTmCD40L and the following primers:

| | |
|---|---|
| 5′- AAC AAG CTC ATT CAG TTC CTG ATC | (SEQ ID NO:37) |
| TCA CTG GTG GGATCC AAC GAC GCA CAA | |
| GCA CCA AAA TC-3′; | |
| 5′- AGC CTT CGG CAG AAG CAT GCC CAG | (SEQ ID NO:38) |
| CAA CAG AAA GTC GTC AAC AAG CTC ATT | |
| CAG TTC CTG-3′; | |
| 5′- AAT GAG GCT CTG TGG CGG GAG GTG | (SEQ ID NO:39) |
| GCC AGC CTT CGG CAG AAG CAT G-3′; | |
| 5′- GAT ATC CTC AGG CTC GAG AAC GAC | (SEQ ID NO:40); |
| GCA CAA GCA CCA AAA GAG AAT GAG GCT | |
| CTG TGG CGG G-3′; | |
| and | |
| 5′- GCGGGCC CGCGGCCGCCGCTAG TCTAGA | (SEQ ID NO:28) |
| GAG TTT GAG TAA GCC AAA AGA TGA | |
| G-3′. | |

HSF1/ΔCtΔTm CD40L with the trimer domain sequence was generated by four rounds of PCR amplification (1^{st} round: primers SEQ ID NOs:37 and 28; 2^{nd} round: primer SEQ ID NOs:38 and 28; 3^{rd} round: primer SEQ ID NOs:39 and 28; 4^{th} round: primer SEQ ID NOs:40 and 28). The HSF1/ΔCtΔTm CD40L encoding DNA was cloned into pcDNA-sig-hMUC-1 restriction sites XbaI (TCTAGA) and Xho I (CTCGAG). The sequence between MUC1 and mCD40L is as follows:

| | |
|---|---|
| LENDAQAPKENEALWREVASFRQKHAQQQKVVN | (SEQ ID NO:41) |
| KLIQFLISLVGSNDAQAPKS, | |

wherein the underlined segment is the trimer sequence which is bonded by the linker LENDAQAPK (SEQ ID NO:42) and NDAQAPKS (SEQ ID NO:43).

In some vectors, a His tag encoding sequence was added to the 3′ end of the sequence encoding ΔCtΔTmCD40L and was generated by PCR using plasmid pDC406-mCD40L (purchased from the American Type Culture Collection) and the following primers:

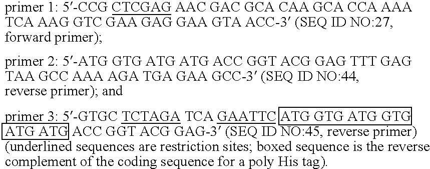

Vector ΔCtΔTmCD40L/His with the His tag sequence was generated by 2 rounds of PCR amplification (1^{st} round: primers 1+2; 2^{nd} round: primer 1+3). The ΔCtΔTmCD40L/His encoding DNA was cloned into pcDNA-sig-ecdhMUC-1 restriction sites XbaI (TCTAGA) and Xho I (CTCGAG).

Primers for amplifying human ΔCtΔTmCD40L+spacer using a human CD40 ligand cDNA template are set forth below.

Human ΔCtΔTmCD40L+ spacer forward primer
(HCD40LSPF) (CD40L sequence italicized):

| | |
|---|---|
| 5′-CCG CTCGAG AAC GAC GCA CAA GCA | (SEQ ID NO:46) |
| CCA AAA TCA GTG TAT CTT CAT AGA AGG | |
| TTG GAC-3′ | |
| Human CD40L reverse primer (HCD40LR) | |
| (CD40L sequence italicized): | |
| 5′-CCC TCTAGA TCA GAG TTT GAG TAA | (SEQ ID NO:47) |
| GCC AAA GGA C-3′ | |

These primers will amplify a ΔCtΔTmCD40L+spacer which encodes 47-261 of human CD40L. The forward primer HCD40LSPF encodes a 10 residue spacer (LENDAQAPKS; single letter code; SEQ ID NO:29) to be located between the tumor antigen and the CD40 ligand (hCD40L) of the transcription unit. PCR performed using the forward and reverse primers (SEQ ID NOs:46 and 47) and plasmid pDC406-hCD40L as the template results in PCR fragment “spacer+ΔCtΔTmCD40L(human),” which is inserted into the plasmid pcDNA-sig-ecdhMUC1 after restriction endonuclease digestion with XbaI (TCTAGA) and Xho I (CTCGAG). The sig-ecdhMUC1/ΔCtΔTmCD40L (human) encoding DNA is cut from the pcDNA3TOPO using HindIII and XbaI restriction enzymes and inserted into pShuttle-CMV (see Murphy et al., Prostate 38: 73-78, 1999) downstream of the CMV promoter. This vector is designated pShuttle sig-ecdhMUC1/ΔCtΔTmCD40L(human). Modification of pShuttle sig-ecdhMUC1/ΔCtΔTmCD40L(human) to include the ecdhMUC1 upstream of the human CD40 ligand sequence is accomplished essentially as described above for the murine CD40 ligand encoding vectors. Thus, the transcription unit sig-ecdhMUC1-ΔCtΔTmCD40L(human) encodes the kappa secretory signal followed by the extracellular domain of human MUC1 followed by a 10 amino acid linker (LENDAQAPKS; SEQ ID NO:29) followed by human CD40 ligand residues 47-261.

In an alternative approach, DNA encoding the human growth hormone signal sequence MATGSRTSLLLAFGLLCLPWLQEGSA (single letter amino acid code) (SEQ ID NO:48) could be used in place of the kappa chain signal sequence.

The recombinant adenoviral vectors were generated using the AdEasy vector system (Stratagene, San Diego, Calif.). Briefly the resulting plasmid pShuttle-sig-ecdhMUC1-ΔCtΔTmCD40L, and other control adenoviral vectors were linearized with Pme I and co-transformed into E. coli strain BJ5183 together with pAdEasy-1, the viral DNA plasmid. Recombinants were selected with kanamycin and screened by restriction enzyme analysis. The recombinant adenoviral construct was then cleaved with Pac I to expose its Inverted Terminal Repeats (ITR) and transfected into 293A cells to produce viral particles. The titer of recombinant adenovirus was determined by the Tissue culture Infectious Dose (TCID₅₀) method.

2. Overcoming Anergy to MUC1 in MUC1 Transgenic Mice

a) Cytokine Production of Adenoviral Infected DCs

Bone marrow derived DCs was harvested from hMUC-.Tg transgenic mice at 48 hours after exposure to the adenoviral vectors. The cells were exposed to vector at MOI 100, and plated in 24-well plates at 2×10⁵ cells/ml. After incubation for 24 hours at 37° C., supernatant fluid (1 ml) was harvested and centrifuged to remove debris. The level of murine IL-12 or IFN-gamma released into the culture medium was assessed by enzyme-linked immunoadsorbent assay (ELISA) using the mouse IL-12 p70 or IFN-gamma R & D Systems kits.

Bone marrow derived DCs contacted with the Ad-sig-ecdmMUC1-ΔCtΔTmCD40L (murine) vector showed significantly increased the levels of interferon gamma and IL-12 cytokines from DCs harvested from the hMUC-.Tg transgenic mice at 48 hours after exposure to the vector. In contrast, virtually no cytokines were detected from restimulated DC's from animals immunized with an adenoviral vector that encoded the extracellular domain of hMUC1 but without fusion to a secretable form of CD40L. These results indicate that the ecdhMUC1/ecdmCD40L (murine) fusion protein forms functional trimers and binds to the CD40 receptor on DCs.

b) Evaluation of Trimer Formation by ecdhMUC1-HSF1-ΔCtΔTmCD40L Fusion Protein Expressed from Ad-sig-ecdhMUC1-HSF1-ΔCtΔTmCD40L-HIS

Trimerization of ecdhMUC1-HSF1-ΔCtΔTmCD40L-HIS fusion protein was evaluated following release from cells transformed with Ad-sig-ecdhMUC1-HSF1-ΔCtΔTmCD40L-HIS vector. The expressed fusion protein was purified from the supernatant of 293 cells exposed to the vector using a His Tag purification kit. Nondenaturing gel electrophoresis showed a molecular weight consistent with trimer formation.

c) Effect of Ad-sig-ecdhMUC1-ΔCtΔTmCD40L Vector Injection on Establishment of MUC1 Expressing Cancer Cells.

hMUC-1.Tg mice injected subcutaneously with the Ad-sig-ecdhMUC1-ΔCtΔTmCD40L (murine) vector were resistant to engraftment by the hMUC1 positive LL2/LL1hMUC1 mouse cancer cells. Control animals not injected with vector were not resistant to the growth of the same cells. Also, hMUC-1.Tg mice injected with the Ad-sig-ecdhMUC1/ecdCD40L (murine) vector were not resistant to engraftment by parental cell line (LL2/LL1), which does not express MUC1.

hMUC-1.Tg mice injected intravenously with ecdhMUC1-ΔCtΔTmCD40L (murine) protein were not resistant to engraftment by the hMUC1 positive LL2/LL1hMUC1 mouse cancer cells. Furthermore, hMUC-1.Tg mice injected with Ad-sig-ecdhMUC1-ΔCtΔTmCD40L (murine) vector lived longer than did control vector injected mice subsequently administered the LL2/LL1hMUC1 cell line.

3. Cellular Mechanisms Underlying Breakdown of Anergy

a) Cytokine Release from Vaccinated vs. Non Vaccinated Mice.

A population of splenic CD8⁺ T lymphocytes was obtained seven days following Ad-sig-ecdhMUC1-ΔCtΔTmCD40L (murine) vector administration was obtained by depleting CD4⁺ T lymphocytes using CD4⁺ antibody coated magnetic beads. The isolated CD8⁺ T lymphocytes released over 2,000 times the level of interferon gamma as did CD8⁺ T cells from MUC-1.Tg mice administered a control vector (without MUC1).

b) Cytotoxicity Assay

Splenic T cells collected from hMUC-1.Tg mice 7 days following administration of Ad-sig-ecdhMUC1-ΔCtΔTmCD40L (murine) vector were cultured with hMUC1 antigen positive LL2/LL1hMUC1 cancer cells in vitro for 7 days. The stimulated splenic T cells were mixed in varying ratios with either the hMUC1 positive LL2/LL1hMUC1 cells or the hMUC1 negative LL2/LL1 cancer cells. The results showed that T cells from Ad-sig-ecdhMUC1-ΔCtΔTmCD40L (murine) vector vaccinated mice were cytotoxic only for the cancer cells expressing hMUC1.

c) Ad-sig-ecdhMUC1-ΔCtΔTmCD40L Vector Injection Overcomes Resistance to Expansion of hMUC1 Specific T Cells.

DCs obtained in vitro from bone marrow cells were exposed to the Ad-sig-ecdhMUC1-ΔCtΔTmCD40L (murine) vector for 48 hours. Splenic CD8⁺ T cells, obtained from hMUC-1.Tg transgenic mice 7 days following no vector injection or subcutaneous injection with the Ad-sig-ecdhMUC1-ΔCtΔTmCD40L (murine) vector, were mixed in a 1/1 ratio with the Ad-sig-ecdhMUC1/ecdCD40L (murine) vector-infected DCs. The ERK1/EK2 proteins, the endpoint of the Ras/MAPK signaling pathway, were phosphorylated in the CD8⁺ T cells isolated from Ad-sig-ecdhMUC1-ΔCtΔTmCD40L vector injected hMUC-1.Tg transgenic mice following 45 minutes of in vitro exposure to Ad-sig-ecdhMUC1-ΔCtΔTmCD40L (murine) vector infected DCs. In contrast no increase in phosphorylation of ERK1 and ERK2 proteins was seen in CD8 positive T cells from unvaccinated hMUC-1.Tg mice. These results demonstrate that CD8 positive T cells from MUC-1.Tg transgenic mice vaccinated with the Ad-sig-ecdhMUC1-ΔCtΔTmCD40L (murine) vector were no longer anergic to MUC1.

4. Production of the Fusion Protein and Vector

The tumor antigen fusion protein was produced directly from an adenoviral vector that carries the expression cassette of the fusion gene encoding the fusion protein. The production cells (e.g. 293 cell line) at 80% confluency in growth medium were infected with the viral vector at the ratio of 10-100 viral particles per cell. The infected cells were further cultured for 48-72 hours, to allow the viral vectors to propagate in the cells and the tumor antigen fusion proteins were expressed in the cells and secreted into culture media. When 70-90% of the cultured cells showed cytopathic effect (CPE), the cells and media were separated and saved. Cell lysates were prepared through 3-time freeze-and-thaw cycles. The viral particles were isolated via the standard procedure (see e.g., PNAS 2003 100:15101-15106; Blood 2004 104:2704-2713). The tumor antigen fusion proteins were purified through affinity chromatograph from the collected cell media.

5. Amplification of the Immune Response by Protein Boosting

The relative value of protein boosting with the tumor antigen fusion protein versus boosting with the adenoviral expression vector was evaluated.

hMUC-1.Tg animals were primed by subcutaneous administration of Ad-K/ecdhMUC1-ΔCtΔTmCD40L vector as described. The protein boost constituted 10 micrograms of ecdhMUC-1/ecdCD40L fusion protein injected subcutaneously. The time of protein boosting and comparison with vector was evaluated in various treatment groups shown in table 2.

TABLE 2
Immunization Schedule

| Testing Group | Week 1 | Week 2 | Week 3 | Week 4 |
|---|---|---|---|---|
| Control | Vector | Vector | Nothing | Nothing |
| Treatment 1 (T1) | Vector | Vector | Protein | Nothing |
| Treatment 2 (T2) | Vector | Vector | Nothing | Protein |
| Treatment 3 (T3) | Vector | Protein | Nothing | Nothing |
| Treatment 4 (T4) | Vector | Nothing | Protein | Nothing |
| Treatment 5 (T5) | Vector | Protein | Nothing | Protein |
| Negative Control | Nothing | Nothing | Nothing | Nothing |

Spleen cells from the different groups were isolated and evaluated by the ELISPOT assay for interferon gamma positivity. As seen in FIG. 3. of U.S. Patent Application Publication US 2005-0226888 (application Ser. No. 11/009,533) titled “Methods for Generating Immunity to Antigen,” two subcutaneous protein injections at a 14 day interval beginning one week after the initial vector injection showed the greatest elevation of the frequency of positive T cells as compared to no treatment or compared with one or two vector injections without protein boost. The next highest elevation of the frequency of interferon gamma positive T cells was with the T3 group (one protein injection 7 days following the initial vector injection).

Cytotoxic T cells development in the various immunization groups was also evaluated; The results are found in FIG. 4 of U.S. Patent Application Publication US 2005-0226888 (application Ser. No. 11/009,533) titled “Methods for Generating Immunity to Antigen.” Spleen cells from the various treatment groups were stimulated in vitro for 5 days with a HMUC-1 positive cell line (LL1/LL2hMUC-1). CD8 T cells were isolated and mixed with the target cells (LL1/LL2hMUC-1) in a 50/1 ratio. Cytotoxic activity generally followed the ELISPOT assay results, with the T5 group showing the greatest increase levels of LL1/LL2hMUC-1 specific cytotoxic T cell activity. The level of cytotoxicity seen with T cells from the T5 group was nine fold that seen with the negative control group.

Serum from the animals in the various treatment groups were evaluated for anti-ecdhMUC1-ΔCtΔTmCD40L specific antibodies in an ELISA. Briefly, microwells coated with the ecdhMUC1-ΔCtΔTmCD40L protein were incubated with test mouse serum, washed and bound mouse antibody identified using a secondary rat anti-mouse antibody conjugated to horseradish peroxidase.

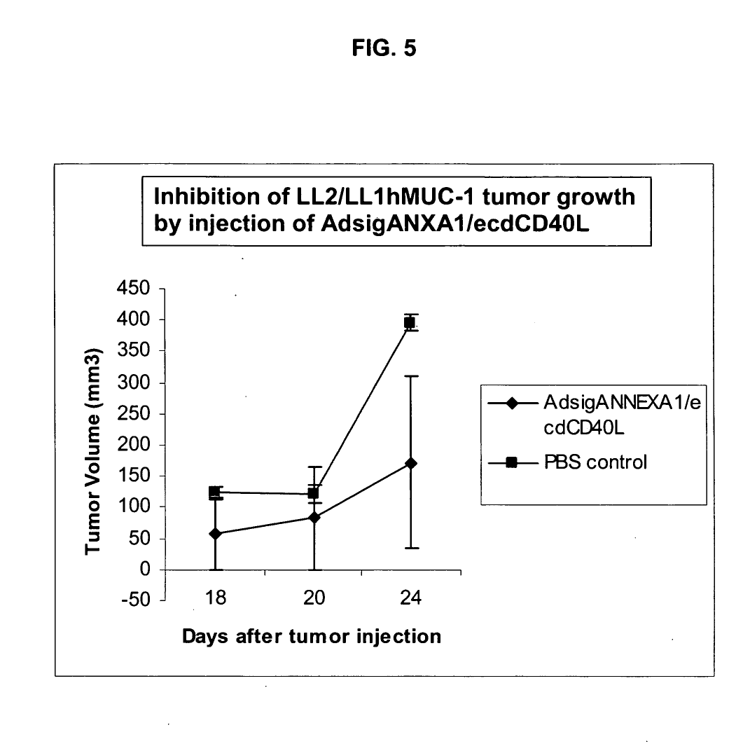

FIG. 5 of U.S. Patent Application Publication US 2005-0226888 (application Ser. No. 11/009,533) titled “Methods for Generating Immunity to Antigen,” shows a dramatic increase in the level of antibodies to the ecdhMUC1-ΔCtΔTmCD40L fusion protein generated by the treatment with one vector injection and two protein injections spaced at a 14 day interval. The increase in the anti-ecdhMUC1-ΔCtΔTmCD40L antibodies following the T5 treatment was 2 fold greater than with any of the other treatment group.

The results from these assays demonstrate that protein boosting is superior to vector boosting in generating cytotoxic T cell activity against tumor antigen expressing cells as well as antibody responses to the tumor antigen. The overall best results with protein boosting were obtained using a single injection of adenoviral expression vector followed one week later with a subcutaneous protein boost, which is repeated two weeks later by another protein boost.

Antibodies in serum from vaccinated hMUC-1.Tg mice were evaluated for binding to cancer biopsy tissue specimens. Tissue microarrays containing normal breast and breast cancer tissue sections were obtained commercially. Tissue was contacted with serum from transgenic mice immunized with Ad-K/ecdhMUC-1//ΔCtΔTm CD40L vector and boosted later with ecdhMUC-1//ΔCtΔTm CD40L protein. The arrays were washed and then exposed to a horseradish peroxidase (HRP) secondary antibody which recognizes mouse IgG antibody. As a control, the serum was exposed first to a hMUC-1 peptide from the antigenic repeat of the hMUC-1 domain (same as used for the protein boost).

Serum from the vaccinated mice bound to the breast epithelial cells from biopsy specimens of cancerous epithelial cells. No binding to the intervening fibroblast or stromal cells were observed. Serum from normal mice showed no reaction.

Serum from hMUC-1.Tg mice vaccinated with the Ad-sig-hMUC-1/ecdCD40L followed by two subsequent administrations of protein sc-hMUC-1/ecdCD40L reacted with biopsy specimens from human prostate cancer on tissue microarray slides.

To determine specificity of the serum generated antibodies for the hMUC-1 repeat, serum from vaccine immunized animals described above was mixed with increasing amounts of a peptide containing the amino acid sequence from the hMUC-1 repeat. The mixture was then applied to the microarray slides and evaluated for reactivity. A peptide with the same amino acids as the hMUC-1 repeat but with the sequence scrambled (“scrambled peptide”) was added to serum from vaccinated animals as a control. The HMUC-1 peptide blocked binding of the antibodies in vaccinated serum to the breast cancer epithelial cells. No blocking was seen for the scrambled peptide. These suggests demonstrate that the vector prime/protein boost vaccination induced a hMUC-1 specific humoral response reactive with MUC-1 expressed by biopsy specimens of human breast cancer epithelial cells.

Tumor immunity in protein boosted mice was evaluated. hMUC-1.Tg animals were primed by subcutaneous administration of Ad-K/ecdhMUC1-ΔCtΔTmCD40L vector as described or were immunized with one or two administrations of the ecdhMUC1-ΔCtΔTmCD40L fusion protein. Animals were then challenged with LL2/LL1hMUC-1 tumor cells.

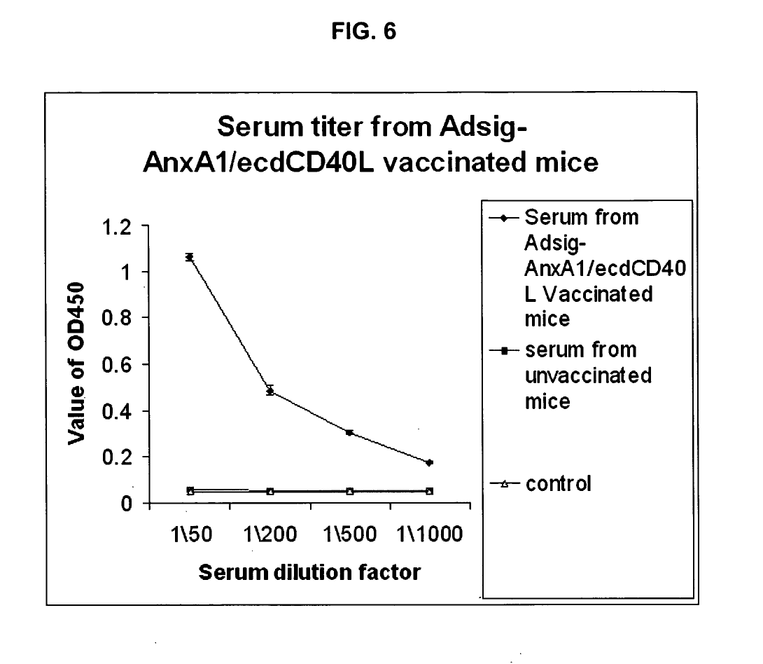

FIG. 6 of U.S. Patent Application Publication US 2005-0226888 (application Ser. No. 11/009,533) titled “Methods for Generating Immunity to Antigen,” shows that mice vaccinated with the Ad-K/ecdhMUC1-ΔCtΔTmCD40L vector survived longer than 120 days (solid bold line), whereas all mice not vaccinated with the Ad-sig-ecdhMUC-1/ecdCD40L vector died by 50 days (broken line). These results show that the vector injections induced a suppression of the growth of the LL2/LL1hMUC-1 cell line in the hMUC-1.Tg mice.

The specificity of tumor growth suppression for the hMUC-1 antigen was evaluated by comparing rejection of the LL2/LL1hMUC-1 cell line (which is positive for the hMUC-1 antigen) with the LL2/LL1 cell line, which is otherwise identical except for the absence of the hMUC-1 antigen. The results showed subcutaneous injection of the adenoviral vector completely suppressed the growth of the LL2/LL1hMUC-1 cell line but did not the same cells which do not express MUC-1.

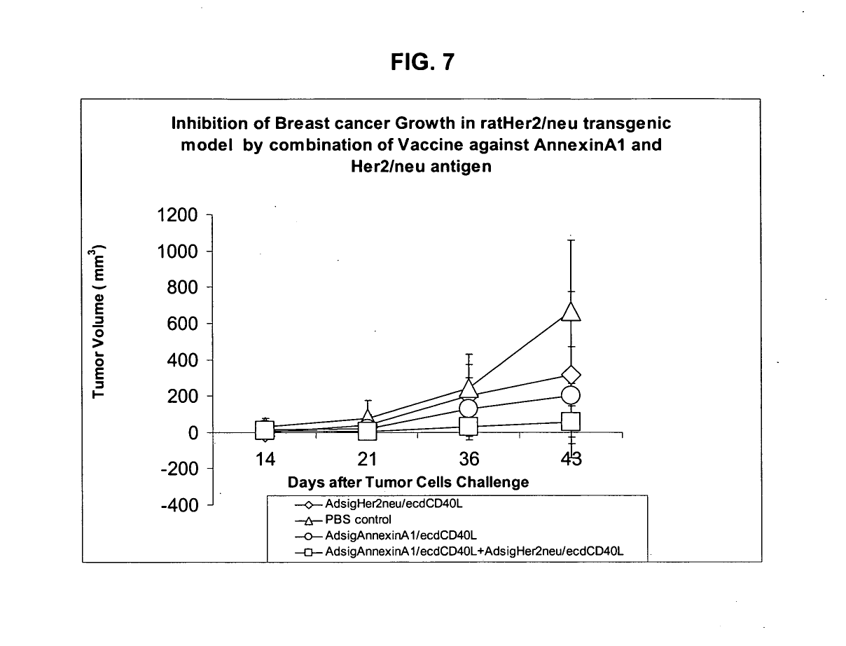

Tumor growth suppression was evaluated using combinations of vector and protein administration. Three combinations of Ad-sig-ecdhMUC-1/ecdCD40L vector and ecdhMUC-1/ecdCD40L protein were administered to hMUC-1.Tg mice before challenge with LL2/LL1hMUC-1 tumor cells. VVV=three Ad-sig-ecdhMUC-1/ΔCtΔTm CD40L vector subcutaneous injections administered on days 1, 7 and 21; PPP=three ecdhMUC-1/ΔCtΔTm CD40L protein subcutaneous injections administered on days 1, 7 and 21; or VPP=a single Ad-sig-ecdhMUC-1/ΔCtΔTm CD40L vector subcutaneous injection followed at days 7 and 21 by ecdhMUC-1/ΔCtΔTm CD40L protein subcutaneous injections. See FIG. 7 of U.S. Patent Application Publication US 2005-0226888 (application Ser. No. 11/009,533) titled “Methods for Generating Immunity to Antigen,” for further details. The mice were challenged one week later with a subcutaneous injection of five hundred thousand LL2/LL1hMUC-1 lung cancer cells, and two weeks later with an intravenous injection of 500,000 LL2/LL1hMUC-1 tumor cells. The size of the subcutaneous tumor nodules at day were measured by caliper at multiple time points to determine the effect of the various vaccine schedules on the growth of the LL2/LL1hMUC-1 cells as subcutaneous nodules. The metasteses were measured by total lung weight following sacrifice.

FIG. 7 of U.S. Patent Application Publication US 2005-0226888 (application Ser. No. 11/009,533) titled “Methods for Generating Immunity to Antigen,” shows that three injections of the fusion protein (PPP) without a preceding Ad-sig-ecdhMUC-1/ecdCD40L vector injection failed to induce complete resistance to the development of the subcutaneous LL2/LL1hMUC-1 tumor. In contrast, the schedule of three successive vector injections (VVV) or one vector injection followed by two protein injections (VPP) completely suppressed the appearance of the subcutaneous LL2/LL1hMUC-1 tumor.

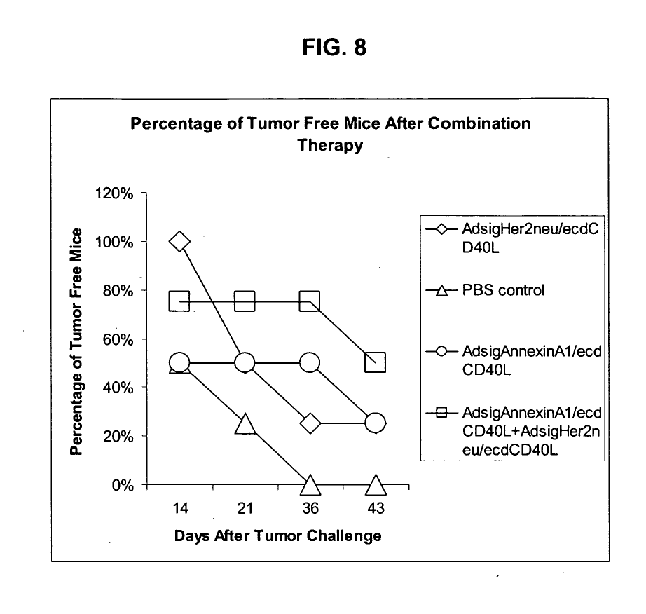

The levels of hMUC-1 specific antibodies in these mice at 63 days following the start of the vaccination were measured (see FIG. 8 of U.S. Patent Application Publication US 2005-0226888 (application Ser. No. 11/009,533) titled “Methods for Generating Immunity to Antigen,”). The schedule of a single vector injection followed by two successive fusion protein boosts (VPP) induced the highest levels of HMUC-1 specific antibodies, schedule VVV was intermediate, and schedule PPP was virtually ineffective. Thus, cancer therapy in these animals related somewhat inversely to the antibody response.

A tumor treatment (post establishment) protocol was also evaluated. In this schedule, subcutaneous tumor (500,000 of the LL2/LL1hMUC-1) was administered on day 1. The three schedules (PPP, VPP and VVV) were accomplished on days 5, 12 and 26. Tumor was administered i.v. on day 35 and tumor development (subcutaneous and lung) evaluated at day 49. Further details are found in the legend to FIG. 9 of U.S. Patent Application Publication US 2005-0226888 (application Ser. No. 11/009,533) titled “Methods for Generating Immunity to Antigen”.

As shown in FIG. 9 of U.S. Patent Application Publication US 2005-0226888 (application Ser. No. 11/009,533) titled “Methods for Generating Immunity to Antigen,” the combination of one vector injection followed by two protein injections (VPP) completely suppressed the growth of established subcutaneous HMUC-1 positive cancer cell tumor. Three successive vector administrations (VVV) had a small therapeutic affect while three successive protein injections (PPP) had little to no effect.

The growth of metastatic lung nodules in the pretreatment and post-treatment (pre-establishment) cancer models is shown in FIG. 10 of U.S. Patent Application Publication US 2005-0226888 (application Ser. No. 11/009,533) titled “Methods for Generating Immunity to Antigen” The pretreatment results in U.S. Patent Application Publication US 2005-0226888 (application Ser. No. 11/009,533) titled “Methods for Generating Immunity to Antigen,” FIG. 10, left hand panel show that three successive fusion protein injections (PPP) did not appear to suppress lung nodule growth. In contrast, schedule VVV and schedule VPP appeared to completely suppress the engraftment of the lung cancer in the lungs of the vaccinated animals.

The post treatment results in of U.S. Patent Application Publication US 2005-0226888 (application Ser. No. 11/009,533) titled “Methods for Generating Immunity to Antigen,” FIG. 10, right hand panel show that the combination of one vector injection followed by two protein injections (VPP) completely suppressed the growth of established lung nodules of the hMUC-1 positive cancer cells. In contrast, three successive vector administrations (VVV) and three successive protein injections (PPP) showed some therapeutic effect but less than for the VPP protocol.

These results suggest that the best overall cancer therapy schedule is the VPP schedule, involving a single injection of Ad-sig-ecdhMUC-1/ecdCD40L vector followed in one week by two successive subcutaneous injections, spaced two weeks apart, of the ecdhMUC-1/ecdCD40L protein. This protocol is characterized by induction of antibody (humoral immunity) and T cell immunity (cellular immunity) to the mucin antigen.

Boosting with ecdMUC-1/ecdCD40L soluble protein versus other soluble proteins following a primary administration of the adenoviral expression vector encoding the same protein was evaluated in hMUC-1.Tg animals challenged with MUC-1 expressing tumor (LL2/LL1hMUC-1 cell line). Animals were boosted with a bacterial extract containing ecdMUC-1/ecdCD40 (from a bacterial host strain infected with Ad-sig-ecdMUC-1/ecdCD40L vector); ecdMUC-1 linked to the keyhole limpet hemocyaninin (KLH), with or without incomplete Freund's adjuvant; PBS; and control bacterial extract (from a bacterial host strain not infected with Ad-sig-ecdMUC-1/ecdCD40L vector). The tumor cells were given 7 days following the completion of the 2nd protein boost. The results shown in FIG. 11 of U.S. Patent Application Publication US 2005-0226888 (application Ser. No. 11/009,533) titled “Methods for Generating Immunity to Antigen,” indicate that boosting with ecdMUC-1/ecdCD40L soluble protein was superior to all other approaches.

6. Construction of Adenoviral Vectors Encoding HPV E7-CD40 Ligand Fusion Protein.

Methods of generating immunity by administering and adenoviral vector expressing a transcription unit fusion protein constituting E7 linked to a secretable form of CD40 ligand was recently reported. (Ziang et al., “An adenoviral vector cancer vaccine that delivers a tumor-associated antigen/CD40-ligand fusion protein to dendritic cells” PNAS (USA) vol. 100(25):15101, 2003).

E7 is a protein encoded by the human papilloma virus which appears on all HPV associated dysplastic and neoplastic cells. The transcription unit included DNA encoding the signal peptide from the HGH gene, upstream of DNA encoding the full-length HPV type 16 E7 protein, consisting of 98 amino acids and having the following amino acid sequence:

| | |
|---|---|
| MHGDTPTLHEYMLDLQPETTDLYCYEQLNDSSEEED | (SEQ ID NO:49) |
| EIDGPAGQAEPDRAHYNIVTFCCKCDSTLRLCVQST | |
| HVDIRTLEDLLMGTLGIVCPICSQKP. | |

The coding sequence for this E7 protein was upstream of the coding sequence of a 10 aa spacer, which was upstream of the coding sequence of the coding sequence of ΔCtΔTmCD40L in the transcription unit.

Construction of an adenoviral vector expressing a transcription unit fusion protein constituting E7 linked to a secretable form of CD40 ligand has been described See, e.g., U.S. Patent Application Publication US 2005-0226888 (application Ser. No. 11/009,533) titled “Methods for Generating Immunity to Antigen,” filed Dec. 10, 2004. This approach is detailed below.

a) Construction of pShuttle-sp-ΔCtΔTmCD40L(No Signal Sequence).

Plasmid pDC406-mCD40L was purchased from the American Type Culture Collection. A pair of PCR primers (SEQ ID NOs:27 and 50) was designed to amplify the mouse CD40 ligand from position 52 to 260 (i.e., without the cytoplasmic and transmembrane domains) and include sequence encoding a linker (indicated as “+spacer”) at the 5′ end of the amplicon.

Forward primer mouse ΔCtΔTmCD40L+spacer (MCD40LSPF) (Xho I recognition site in bold and underlined; spacer sequence underlined (includes the Xho I site); CD40L sequence italicized):

| 5′-CCG CTCGAG AAC GAC GCA CAA GCA | (SEQ ID NO:27) |
|---|---|
| CCA AAA TCA AAG GTC GAA GAG GAA GTA | |
| AAC-3′ | |
| and | |
| reverse primer mouse CD40L (MCD40LR) | |
| (XbaI recognition site in bold and | |
| underlined) | |
| 5′-CCC TCTAGAATCAGAGTTTCACTAAGCCAA- | (SEQ ID NO:50) |
| 3′ | |

The forward primer MCD40LSPF encoded a 10 residue spacer (LENDAQAPKS; single letter code; SEQ ID NO:29) to be located between the tumor antigen and the CD40 ligand (mCD40L) of the transcription unit. PCR was performed using the forward and reverse primers (SEQ ID NOs:27 and 50) and plasmid pDC406-mCD40L as the template under the following conditions: hold 3 min at 94° C.; cycle 94° C. for 45 sec, 55° C. for 45 sec, 72° C. for 70 sec (30 cycles); hold 7 min at 72° C.; and hold at 4° C. This PCR resulted in a fragment “spacer+ΔCtΔTmCD40L,” which was inserted into the plasmid pShuttle-CMV (Murphy et al. Prostate 38:73-8, 1999) after restriction endonuclease digestion with XbaI (TCTAGA) and Xho I (CTCGAG). This vector was designated pShuttle-sp-ΔCtΔTmCD40L(no signal sequence).

A vector was produced that was otherwise the same except that it encoded full length CD40L rather than the truncated form. This vector was made using a CD40 forward primer that annealed to the starting codons of murine CD40L. This vector was designated pShuttle-mCD40L (no signal sequence).

b) Construction of pShuttle-E7-sp-ΔCtΔTmCD40L(No Signal Sequence).

pShuttle-E7-ΔCtΔTmCD40L (no signal sequence) was prepared by inserting HPV-16 E7 upstream of the CD40 ligand sequence as follows: sequence encoding the full-length HPV-16 E7 protein was obtained by PCR amplifying from the HPV viral genome using the following primers:

HPV 16 E7 forward primer (SEQ ID NO:51, Not I site
in bold and underlined)
5′-ATTT GCGGCCGC TGTAATCATGCATGGAGA-3;′
HPV E7 reverse primer (SEQ ID NO:52, Xho I site in
bold and underlined)
5′-CC CTCGAG TTATGGTTTCTGAGAACAGAT-3.′

PCR was performed using the above primers and the HPV 16 viral genome as template under the following conditions: hold 3 min at 94° C.; cycle 94° C. for 40 sec, 58° C. for 40 sec, 72° C. for 40 sec (30 cycles); hold 7 min at 72° C.; and hold at 4° C. The resulting amplicon was HPV 16 E7 encoding DNA with Not I and Xho 1 restriction sites at the 5′ and 3′ ends, respectively. The E7 DNA was inserted into the pShuttle-sp-ΔCtΔTmCD40L(no signal sequence) vector between the CMV promoter and directly 5′ to the spacer of the spacer-ΔCtΔTmCD40L sequence using the Not I (GCGGCCGC) and Xho I (CTCGAG) restriction sites. The resulting plasmid was designated pShuttle-E7-ΔCtΔTmCD40L(no signal sequence).

c) Construction of pShuttle-HGH/E7-sp-ΔCtΔTmCD40L.

The pShuttle-E7-sp-ΔCtΔTmCD40L(no signal sequence) vector was used for insertion of the HGH signal sequence, upstream of E7 to generate HGH/E7-sp-ΔCtΔTmCD40L, described as follows.

DNA encoding the human growth hormone signal sequence MATGSRTSLLLAFGLLCLPWLQEGSA (single letter amino acid code) (SEQ ID NO:48) was prepared by annealing phosphorylated oligonucleotides (SEQ ID NOs:53 and 54) to generate the full 26 amino acid HGH sequence with Bgl II and Not I overhangs. Growth hormone signal upper strand (coding sequence in italics):

| | |
|---|---|
| Growth hormone signal upper strand | |
| (coding sequence in italics): | |
| 5′-GATCT CCACC ATG GCT ACA GGC TCC | (SEQ ID NO:53) |
| CGG ACG TCC CTG CTC CTG GCT TTT GGC | |
| CTG CTC TGC CTG CCC TGG CTT CAA GAG | |
| GGC AGT GCC GGC -3′; | |
| Growth hormone signal lower strand: | |
| 3′-A GGTGG TAC CGA TGT CCG AGG GCC | (SEQ ID NO:54) |
| TGC AGG GAC GAG GAC CGA AAA CCG GAC | |
| GAG ACG GAC GGG ACC GAA GTT CTC CCG | |
| TCA CGG CCGCCGG -5′. | |

Synthetic HGH signal sequence was prepared by annealing the above upper and lower strand oligos. The oligos were dissolved in 50 μl H₂O (about 3 mg/ml). 1 μl of each oligo (upper and lower strand) was added to 48 μl annealing buffer (100 mM potassium acetate, 30 mM HEPES-KOH pH 7.4, and 2 mM Mg-acetate) incubated for 4 minutes at 95° C., 10 minutes at 70° C. and slowly cooled to about 4° C. The annealed DNA was phosphorylated using T4 PNK (polynucleotide kinase) under standard conditions.

The HGH signal sequence with Bgl II and Not I overhangs was inserted via Bgl II and Not I into pShuttle-E7-sp-ΔCtΔTmCD40L(no signal sequence) to yield pShuttle-HGH/E7-sp-ΔCtΔTmCD40L. Thus, the transcription unit HGH/E7-sp-ΔCtΔTmCD40L encodes the HGH secretory signal followed by the full length HPV type 16 E7 followed by a 10 amino acid linker with (LENDAQAPKS; SEQ ID NO:29) followed by murine CD40 ligand residues 52-260.

d) Construction of pShuttle-K/E7-sp-ΔCtΔTmCD40L

A transcription unit that included DNA encoding the signal sequence of the mouse IgG kappa chain gene upstream of DNA encoding the full length HPV type 16 E7 protein (“K/E7”) was generated by PCR using HPV16 plasmid and the following primers:

| | |
|---|---|
| (primer 1) | |
| 5′- CCACC ATG GAG ACA GAC ACA CTC | (SEQ ID NO:22) |
| CTG CTA TGG GTA CTG CTG-3′ | |
| (primer 2) | |
| 5′- TC CTG CTA TGG GTA CTG CTG CTC | (SEQ ID NO:23) |
| TGG GTT CCA GGT TC-3′ | |
| (primer 3) | |
| 5′- TG CTC TGG GTT CCA GGT TCC ACT | (SEQ ID NO:55) |
| GGT GAC ATG CAT G-3′; | |
| (primer 4) | |
| 5′- TGG GTT CCA GGT TCC ACT GGT GAC | (SEQ ID NO:56) |
| ATG CAT GGA G AT ACA CCT AC-3′; | |
| and | |
| (primer 5) | |
| 5′- CCG CTC GAG TGG TTT CTG AGA ACA | (SEQ ID NO:57) |
| GAT GGG GCA C -3.′ | |

K/E7 with the upstream kappa signal sequence was generated by four rounds of PCR amplification (1^{st} round: primers 4+5; 2^{nd} round: add primer 3; 3^{rd} round: add primer 2; 4^{th} round: add primer 1). The K/E7 encoding DNA was cloned into the pcDNA™ 3.1 TOPO vector (Invitrogen, San Diego, Calif.) forming pcDNA-K/E7.

A DNA fragment that contained coding sequence for the 10 aa spacer upstream of mouse CD40 ligand from which the transmembrane and cytoplasmic domain had been deleted (−sp-ΔCtΔTmCD40L) was generated from a mouse CD40 ligand cDNA plasmid, pDC406-mCD40L (American Type Culture Collection), using the following PCR primers:

| | |
|---|---|
| 5′-CCG CTCGAG AAC GAC GCA CAA GCA | (SEQ ID NO:58) |
| CCA AAA AGC AAG GTC GAA GAG GAA GTA | |
| AAC CTT C-3′; | |
| and | |
| 5′-CGCGCCGCGCGCTAG TCTAGA GAGTTTGAG | (SEQ ID NO:59) |
| TAAGCCAAAAGATGAG-3′ | |
| (High Fidelity PCR kit, Roche). | |

Fragment sp-ΔCtΔTmCD40L was digested with Xba I and XhoI restriction endonucleases and then ligated into pcDNA-K/E7. The K/E7-sp-ΔCtΔTmCD40L fragment was cut from the pcDNA vector and inserted into the pShuttle plasmid using Hind III and Xba I sites (pShuttle K/E7-sp-ΔCtΔTmCD40L). Thus, the K/E7-sp-ΔCtΔTmCD40L fragment includes the kappa chain secretory signal followed by the full length HPV type 16 E7 followed by a 10 amino acid linker (LENDAQAPKS; SEQ ID NO:29) followed by murine CD40 ligand residues 52-260.

e) Construction of pShuttle-HGH/E7-CD40L.

Adenoviral vector encoding a fusion protein with E7 upstream of full length mouse CD40L (with no intervening linker) was made using primers to amplify full length mouse CD40L using PCR. The following primers were used:

| | |
|---|---|
| forward primer: | |
| 5′- GAGAC CTCGAG CAGTCA GC ATGATAGA | (SEQ ID NO:60) |
| AACATACAGC CAACCTTCCC-3′; | |
| reverse primer: | |
| 5′-CGCGCCGCGCGC CCC TCTAGA TCA GAG | (SEQ ID NO:61) |
| TTT GAG TAA GCC AAA AGA TGA G-3′. | |

Amplified DNA was initially subcloned into the pcDNA3-K/E7 vector with Xba I and XhoI restriction endonucleases. The full length CD40L gene or ΔCtΔTmCD40L was directionally cloned into the pShuttle plasmid with the Hind III and Xba I sites.

f) Construction of pShuttle-HGH/E7-sp-ΔCtΔTmCD40L(Human).

A vector encoding an E7/human CD40 ligand fusion protein (pShuttle-HGH/E7-sp-ΔCtΔTmCD40L(human)) is described as follows.

Primers for amplifying human ACtATMCD40L+spacer using a human CD40 ligand cDNA template are set forth below.

Human ΔCtΔTmCD40L+spacer forward primer (HCD40LSPF) (XhoI site in bold and underlined; spacer underlined (includes XhoI site); CD40L sequence italicized):

| | |
|---|---|
| 5′- CCG CTCGAG AAC GAC GCA CAA GCA | (SEQ ID NO:46) |
| CCA AAA TCA GTG TAT CTT CAT AGA AGG | |
| TTG GAC -3′; | |

Human CD40L reverse primer (HCD40LR):

| | |
|---|---|
| 5′-CCC TCTAGA TCAGAGTTTGAGTAAGCCAAAG | (SEQ ID NO:47) |
| GAC-3′. | |

PCR is performed using the above primers and the plasmid pDC406-hCD40L as template under the following conditions: hold 3 min at 94° C.; cycle 94° C. for 45 sec, 52° C. for 45 sec, 72° C. for 70 sec (30 cycles); hold 7 min at 72° C.; and hold at 4° C. This amplification results in the “−sp-ΔCtΔTmCD40L(human)” fragment, which encodes 47-261 of human CD40L and an amino terminal 10 aa spacer. The forward primer HCD40LSPF encodes a 10 residue spacer (LENDAQAPKS; single letter code; SEQ ID NO:29) to be located between the tumor antigen and the CD40 ligand (hCD40L) of the transcription unit. The “sp-ΔCtΔTmCD40L(human)” fragment is then inserted into the plasmid pShuttle-CMV (Murphy G P, et al. Prostate 38: 73-78 (1999)) after restriction endonuclease digestion with XbaI (TCTAGA) and Xho I (CTCGAG). This vector is designated pShuttle-sp-ΔCtΔTmCD40L(human)(no signal sequence). Modification of pShuttle-sp-ΔCtΔTmCD40L(human)(no signal sequence) to include the HPV-16 E7 upstream of the human CD40 ligand sequence is accomplished essentially as described above for the murine CD40 ligand encoding vectors. The resulting plasmid is designated pShuttle-E7-sp-ΔCtΔTmCD40L(human)(no signal sequence) and is used for insertion of the HGH signal sequence upstream of E7 to generate HGH/E7-sp-ΔCtΔTmCD40L(human). Thus, the transcription unit HGH/E7-sp-ΔCtΔTmCD40L(human) encodes the HGH secretory signal, followed by the full length HPV type 16 E7, followed by a 10 amino acid linker (LENDAQAPKS; SEQ ID NO:29) followed by human CD40 ligand residues 47-261.

7. Construction of Adenoviral Vectors Encoding ratHER2(Neu)/CD40L

The overexpression of the Her-2-Neu (H₂N) growth factor receptor in 30% of breast cancers is associated with increased frequency of recurrence after surgery, and shortened survival. Mice transgenic for the rat equivalent of HER2 (“H₂N” or “rH2N”) gene and therefore tolerant of this gene (Muller et al., Cell 54: 105-115, (1988); Gut et al. Proc. Natl. Acad. Sci. USA 89: 10578-10582, (1992)) were used as experimental hosts for evaluating immunity in the Ad-sig-rH2N/ecdCD40L vector. In this model, the mouse is made transgenic for a normal unactivated rat Her-2-Neu gene under the control of a mammary specific transcriptional promoter such as the MMTV promoter. The MMTV promoter produces overexpression of a non-mutant rat Her-2-Neu receptor, which is analogous to what occurs in human breast cancer. This model produces palpable tumor nodules in the primary tissue (the breast) at 24 weeks as well as pulmonary metastases at 32 weeks. The development of breast cancer occurs spontaneously. The cancer begins focally as a clonal event in the breast epithelial tissue through a step-wise process (Id.). Dysplasia can be detected by 12 weeks. Palpable tumors in the mammary glands can be detected at 25 weeks, and metastatic breast cancer in the lung can be demonstrated in 70% of mice by 32 weeks (Id.).

Ad-sig-rH2N/ecdCD40L vector was subcutaneously administered to transgenic animals one or two times at 7 day intervals to test if an immune response could be induced against the rat Her-2-Neu antigen. Two subcutaneous injections of the Ad-sig-rH2N/ecdCD40L vector induced complete resistance to the growth of the N₂O₂ (rH2N positive) mouse breast cancer cell line, whereas one subcutaneous injection of the same vector did not induce sufficient immune response to completely suppress the growth of the rH2N positive N₂O₂ cell line. ELISPOT assays showed that the administration of two subcutaneous injections of the Ad-sig-rH2N/ecdCD40L vector 7 days apart induced levels of rH2N specific T cells in the spleens of vaccinated mice which were 10 times higher than the levels of rH2N specific T cells induced in mice following one injection of the Ad-sig-rH2N/ecdCD40L vector. Finally, the immune resistance induced against the NT2 cells by the Ad-sig-rH2N/ecdCD40L vector prime vaccination was better than the response obtained in transgenic animals vaccinated with irradiated cytokine positive tumor cells (mitomycin treated NTW cells which had been transfected with a GMCSF transcription unit).

The rH2N specific antibody levels were also measured in mice vaccinated with one or two subcutaneous injections of the Ad-sig-rH2N/ecdCD40L vector. As shown below in FIG. 11, the levels of the rH2N specific antibody levels were higher following two subcutaneous injections than following a single subcutaneous injection of the Ad-sig-rH2N/ecdCD40L vector.

8. Construction of Adenoviral Vectors Encoding hHER2/CD40L

An adenoviral vector encoding sig ecdhHER2/CD40L is prepared as follows. Total RNA is extracted from the HER2/Neu-expressing human cancer cell line SK-BR-3 and used as a template for cDNA synthesis using the SuperScript™ First Strand Synthesis System (Invitrogen San Diego, Calif.). The resulting cDNA is used as template in the following PCR amplification. The mouse IgG kappa chain METDTLLLWVLLLWVPGSTGD (single letter amino acid code) (SEQ ID NO:21) is prepared by PCR amplification (using the primers as set forth in SEQ ID NOs:22, 23, and 62) in conjunction with the amplification of HER2 (using the primers set forth in SEQ ID NOs:63 and 64) to generate the full 21 amino acid mouse IgG kappa chain signal sequence (the start codon “ATG” is shown bolded in SEQ ID NO:22).

| | |
|---|---|
| 5′-CCACC ATG GAG ACA GAC ACA CTC CTG | (SEQ ID NO:22) |
| CTA TGG GTA CTG CTG-3′; | |
| 5′- TC CTG CTA TGG GTA CTG CTG CTC | (SEQ ID NO:23) |
| TGG GTT CCA GGT TC-3′; | |
| 5′- TG CTC TGG GTT CCA GGT TCC ACT | (SEQ ID NO:62) |
| GGT GAC GAA CTC -3′; | |

the forward primer for the human HER2 extra-
cellular domain

| | |
|---|---|
| 5′- TCC ACT GGT GAC GAACTCACCTACCTGC | (SEQ ID NO:63) |
| CCACCAATGC-3′; | |
| and | |

the reverse primer for the human HER2 extra-
cellular domain

| | |
|---|---|
| 5′- GGAGCTCGAG GGCTGGGTCCCCATCAAAGCT | (SEQ ID NO:64) |
| CTC-3′. | |

sig-ecdhHER2 with the upstream kappa signal sequence is generated by four rounds of PCR amplification (1^{st} round: primers SEQ ID NOs:63 and 64; 2 round: primer SEQ ID NOs:62 and 64; 3^{rd} round: primer SEQ ID NOs:23 and 64; 4 h round: primer SEQ ID NOs:22 and 64) under the following PCR conditions: hold 3 min at 94° C.; cycle 94° C. for 45 sec, 53° C. for 50 sec, 72° C. for 60 sec (30 cycles); hold 7 min at 72° C.; and hold at 4° C. The sig-ecdhHER2 encoding DNA can be cloned into the pcDNA™ 3.1 TOPO vector (Invitrogen, San Diego, Calif.) forming pcDNA-sig-ecdhHER2. The additional cloning steps described for the MUC-1/CD40 ligand expression vector are also applicable for the HER2/CD40 ligand expression vector.

This region HER2 extracellular domain to be fused to CD40 ligand contains two CTL epitopes; one is an HLA-A2 peptide, K I F G S L A F L (SEQ ID NO:65) representing amino acids 369-377. This peptide elicited short-lived peptide-specific immunity in HER2 expressing cancer patients. See Knutson et al., Immunization of cancer patients with a HER-2/neu, HLA-A2 peptide, Clin Cancer Res. 2002 May; 8(5):1014-8p369-377. The second epitope is E L T Y L P T N A S (SEQ ID NO:66) (HER2 residues 63-71) also was useful in generating immunity to HER2 expressing tumor cells. See Wang et al. Essential roles of tumor-derived helper T cell epitopes for an effective peptide-based tumor vaccine, Cancer Immun. 2003 Nov. 21; 3:16. The region of the HER2 ecd also includes a B cell epitope P L H N Q E V T A E D G T Q R C E K C S K P C (SEQ ID NO:67)(HER2 positions 316-339). See Dakappagari et al., Chimeric multi-human epidermal growth factor receptor-2 B cell epitope peptide vaccine mediates superior antitumor responses, J Immunol. 2003 Apr. 15; 170(8):4242-53.

9. Construction of Ad-sig-AnxA1/ecdCD40L Expression Vector

The transcription unit, sig-AnxA1/ecdCD40L, of the adenoviral vector encodes a signal sequence (from an Ig kappa chain) followed by a 167 amino acid fragment of mouse annexin A1 (GenBank Accession No. NM_{—}010730) having the following sequence:

| | |
|---|---|
| PAQFDADELRGAMKGLGTDEDTLIEILTTRSNEQIRE | (SEQ ID NO:5) |
| INRVYREELKRDLAKDITSDTSGDFRKALLALAKGDR | |
| CQDLSVNQDLADTDARALYEAGEIRKGTDVNVFTTIL | |
| TSRSFPHLRRVFQNYGKYSQHDMNKALDLELKGDIEK | |
| CLTTIVKCATSTPAFFAEK. | |

The annexin A1 fragment was connected via a linker to a fragment of the CD40 ligand (human or mouse) which contains the extracellular domain without the transmembrane or cytoplasmic domains. The fusion protein was engineered to be secreted from vector infected cells by the addition of the kappa chain signal sequence to the amino-terminal end of the fusion protein.

An adenoviral vector encoding sig-mAnxA1/CD40L was prepared as follows. Total RNA was extracted from mouse endothelial cells from a tumor nodule and cDNA was synthesized from total RNA using the SuperScript™ First-Strand Synthesis System (Invitrogen, San Diego, Calif.). The resulting cDNA was used as the template in the following PCR amplification. The mouse IgG kappa chain METDTLLLWVLLLWVPGSTGD (single letter amino acid code) (SEQ ID NO:21) was prepared by PCR amplification (using the primers set forth in SEQ ID NOs:22, 23 and 68) in conjunction with amplification of mouse annexin A1 (using the primers set forth in SEQ ID NOs:69 and 70) to generate the full 21 amino acid mouse IgG kappa chain signal sequence (the start codon “ATG” is shown bolded in SEQ ID NO:22) fused to the N-terminus of the annexin A1 fragment.

| | |
|---|---|
| Primer 1: | |
| 5′-CCACC ATG GAG ACA GAC ACA CTC CTG | (SEQ ID NO:22) |
| CTA TGG GTA CTG CTG-3′; | |
| primer 2: | |
| 5′- TC CTG CTA TGG GTA CTG CTG CTC | (SEQ ID NO:23) |
| TGG GTT CCA GGT TC-3′; | |
| primer 3: | |
| 5′- TG CTC TGG GTT CCA GGT TCC ACT | (SEQ ID NO:68) |
| GGT GAC CCAGCT -3′; | |

primer 4 (forward primer for the mouse annexin A1
fragment):

| | |
|---|---|
| 5′-TCCACTGGTGACCCAGCTCAGTTTGATGCAGAT | (SEQ ID NO:69) |
| G-3′, | |
| and; and | |

primer 5 (reverse primer for the mouse annexin A1
fragment):

| | |
|---|---|
| 5′- GGAGCTCGAGCTTCTCGGCAAAGAAAGCTGGA | (SEQ ID NO:70) |
| GTG-3′. | |

The sig-mAnxA1 nucleic acid sequence encoding the kappa signal sequence upstream of the annexin A1 fragment was generated by four rounds of PCR amplification (1^{st} round: primers 4 and 5; 2^{nd} round: primers 3 and 5; 3^{rd} round: primers 2 and 5; 4^{th} round: primers 1 and 5). The PCR conditions were as follows 94° C. for 1 min, 57° C. for 1 min, and 72° C. for 45 s for 30 cycles, followed by a 10 min incubation at 72° C. The sig-mAnxA1 encoding DNA was cloned into the pcDNA™ 3.1 TOPO vector (Invitrogen, San Diego, Calif.) forming pcDNA-sig-AnxA1. The additional cloning steps described for the MUC-1/CD40 ligand expression vector were also applicable for the mAnxA1/CD40 ligand expression vector.

10. Production of the Ad-sig-AnxA1/ecdCD40L Fusion Protein and Vector

The Ad-sig-AnxA1/ecdCD40L viral particles were produced by the following method. The production cells (293 cell line) at 80% confluency in growth medium were infected with the Ad-sig-AnxA1/ecdCD40L viral vector at the ratio of 10-100 viral particles per cell. The infected cells were further cultured for 48-72 hours, to allow the viral vectors to propagate in the cells and the fusion protein to be expressed in the cells and secreted into culture media. When the cultured cells showed 70-90% cytopathic effect (CPE), the cells and media were separated and saved. Cell lysates were prepared through 3-time freeze-and-thaw cycles. The viral particles were isolated via the standard procedure (see e.g., PNAS 2003 100:15101-15106; Blood 2004 104:2704-2713).

The AnxA1/ecdCD40L fusion protein was produced by the following method. The AnnexA1/ecdCD40L cDNA was amplified from the template Ad-sig-AnxA1/ecdCD40L with the following primers:

5′- AA CCA TCA CTC TTC TGG T AGATCT CCAGCTCAGTTTGA
TGCAGATGAACTC-3′
(forward primer, SEQ ID NO:71, XcmI restriction
site underlined)
and
5′- CCGCTCGAGGCAGATATCTCAGAGTTTGAGTAAGCCAAAAGATGA
G-3′
(reverse primer, SEQ ID NO:72, XhoI restriction
site underlined).

The product was inserted into the pTriEx-2 hygro Vectors (Novagen) following XcmI and XbaI digestion. Competent cells (Rosetta™ cells, Novagen Inc.) were transformed with the resulting plasmid. Following incubation of the cells in IPTG supplemented medium for 4 hours, a cell lysate was prepared using the Cellytic™ B Plus Kit (Sigma). The AnnexA1/ecdCD40L protein was purified from the soluble fraction by HIS-select Nickel Affinity Gel (Sigma). Then, the protein was concentrated and desalted by centrifugation through an Ultrafree-15 Biomax-50 filter (Millipore) and eluted with PBS.

11. Suppression of Tumor Growth by Ad-sig-AnxA1/ecdCD40L Vector with Fusion Protein Boost

Annexin 1A (Anx1A) protein is present on the luminal surface of the endothelial cells of tumor vasculature but was not detectable on the luminal surface of the vascular endothelial cells of normal tissues. We therefore decided to test if one subcutaneous injections of the Ad-sig-Anx1A/ecdCD40L vector followed by 2 protein boosts (7 days apart) of the Anx1A/ecdCD40L fusion protein would suppress the growth of the hMUC-1 positive LL2/LL1hMUC-1 cell line in hMUC-1.Tg mice. Mice (4 per group) which were transgenic for the hMUC-1 genes were vaccinated via sc injection with 1×10⁸ pfu of the Ad-sig AnnexA1/ecdCD40L, followed by two protein boosts. One week after the last vaccination, hMUC-1.Tg mice were challenged by sc injection of 1×10⁵ hMUC-1 positive cancer cells/mouse.

As shown by the experimental results shown in FIG. 5, this vector induces regression of the hMUC-1 positive cells in hMUC-1.Tg mice. This suggests that the Ad-sig-TAA/ecdCD40L vaccine strategy can induce an immune response against endothelial cells of tumor vascular endothelial cells.

12. Specificity of the Immune Response Generated by Ad-sig-AnxA1/ecdCD40L Vector with Fusion Protein Boost

In order to test if the immune response generated by the Ad-sig-AnxA1/ecdCD40L vector was directed against the Annexin A1 antigen, plasma was taken from a mouse, which had been vaccinated with the Ad-sig-AnxA1/ecdCD40L vector and two subsequent protein boosts of the AnxA1/ecdCD40L fusion protein, and tested by ELISA assay for the presence of antibodies against the Annexin A1 antigen.

Plasma was collected from test mice before vaccination and 1 week after the last protein boost. Plates were coated with annexin A1 antigen overnight. Nonspecific binding was blocked by a 1-h incubation with 5% BSA in PBS. Mouse plasma pools (1:50/1:200/1:500/1:1000 dilutions) were added for 2 h at room temperature. The plates were washed and HRP-conjugated goat anti-mouse IgG (Jackson ImmunoResearch Laboratories, Inc) was added. Bound antibody was detected with o-phenylenediamine dihydrochloride (Sigma). The reaction was stopped with 50 μL of 2 N H₂SO₄, and the absorbance at 450 nm was evaluated with an automatic ELISA reader.

As shown in FIG. 6, antibodies which bind annexin A1 were induced in the serum of the Ad-sig-AnxA1/ecdCD40L vaccinated hMUC-1.Tg mice.

13. Localization of Binding of Antibodies Against AnxA1

In order to specifically test if these antibodies are binding to the tumor vasculature, multiparameter fluorescence in situ confocal microscopy was carried out on tissue samples taken from mice vaccinated with the Ad-sig-AnxA1/ecdCD40L vector and two subsequent protein boosts of the AnxA1/ecdCD40L fusion protein. It was observed that antibodies against annexin A1 induced by the Ad-sig-AnxA1/ecdCD40L vector vaccination bound to the tumor vasculature as shown by the coincidence (yellow color) of the anti-CD31 antibodies (labeled red) against the tumor vasculature and the binding of the Annexin A1 antibody (labeled green).

In a further study, the binding of serum from the bloodstream of Ad-sig-AnxA1/ecdCD40L vaccinated mice to paraffin embedded formalin fixed sections of various tissues was evaluated. Staining was observed for the vasculature of tumor tissue, but not for vessels of normal lung, brain, or kidney.

14. Tumor growth Suppression by the Combination of the Ad-sig-AnxA1/ecdCD40L and Ad-sig-TAA/ecdCD40L Vaccines

In this study, tumor growth suppression by the concomitant administration of an Ad-sig-TVECA/ecdCD40L vaccine and an Ad-sig-TAA/ecdCD40L vaccine was compared to tumor growth suppression when either vaccine is administered alone. Test mice were vaccinated with the combination of the Ad-sig-AnxA1/ecdCD40L TVECA vaccine and the Ad-sig-rH2N/ecdCD40L TAA vaccine (4 mice) or each of the individual vaccines alone (Ad-sig-rH2N/ecdCD40L alone or Ad-sig-AnxA1/ecdCD40L alone). Seven days after the administration of the vaccine, the test mice were injected subcutaneously with 500,000 of the N₂O₂ breast cancer cells which are positive for the rH2N gene and AnxA1 negative. As shown below in FIG. 7, the effect of the combination of the Ad-sig-rH2N/ecdCD40L and the Ad-sig-AnxA1/ecdCD40L vaccines (open squares) was greater than the effect of either vaccine alone (open diamonds or open circles). The number of mice that remained tumor-free. As shown below in FIG. 8, the mice vaccinated with a combination of the Ad-sig-rH2N/ecdCD40L and the Ad-sig-AnxA1/ecdCD40L tumor vascular targeting vaccine vector showed the highest percentage of mice which remained tumor-free, whereas mice vaccinated with either the Ad-sig-hMUC-1/ecdCD40L or the Ad-sig-rH2N/ecdCD40L showed a much lower percentage of tumor-free mice.

15. Construction of Her2/Neu+AnnexA1 Chimeric Vector

Using the pcDNA-sig-ecdhHER2 vector from Example 8 as template, Her2/Neu cDNA is amplified using the following primers:

5′-CCACC ATG GAG ACA GAC ACA CTC CTG CTA TGG GTA
CTG CTG-3′
(forward primer, SEQ ID NO:22)
and
5′-CCG CTGGAG GCA GATATC GGCTGGGTCCCCATCAAAGCTCT
C-3′
(reverse primer, SEQ ID NO:73, Eco RV site in bold
and underlined).

The K/Her2/Neu encoding DNA is cloned into the pcDNA™ 3.1 TOPO vector (Invitrogen, San Diego, Calif.) forming pcDNA-K/Her2/Neu.

To obtain the annexin A1 fragment, total RNA is extracted from mouse endothelial cells from a tumor nodule, cDNA is synthesized from total RNA using the SuperScript™ First-Strand Synthesis System (Invitrogen, San Diego, Calif.). The resulting cDNA is used immediately to amplify the annexin A1 fragment by PCR using the following primers:

5′- GCA GATATC GCAATGGTATCAGAATTCCTCAAG -3′
(annexin A1 forward primer, SEQ ID NO:74, Eco RV
site in bold and underlined)
and
5′- CCG CTCGAG GAGTTTTTAGCAGAGCTAAAACAACC -3′
(annexin A1 reverse primer, SEQ ID NO:75, Xho I
site in bold and underlined).

The annexin A1 fragment-encoding DNA is cloned into the pcDNA™ 3.1 TOPO vector (Invitrogen, San Diego, Calif.) forming pcDNA-AnnexA1. Annexin A1-encoding DNA is cut from pcDNA-AnnexA1 by Eco RV and Xho I and inserted into pcDNA-K/Her2/Neu with same sites.

“spacer+ΔCtΔTmCD40L(human)” is inserted into the plasmid pcDNA-sig-Her2/Neu+AnnexA1 after restriction endonuclease digestion with Xba I (TCTAGA) and Xho I (CTCGAG). The sig-Her2/Neu+AnnexA1/ΔCtΔTmCD40L(human) encoding DNA was cut from the pcDNA3TOPO using Hind III-Xba I restriction enzymes and inserted into pShuttle-CMV downstream of the CMV promoter. This vector is designated pShuttle-sig-Her2/Neu+AnnexA1/ΔCtΔTmCD40L(human).

16. Construction of Human Annexin A1/Human CD40 Ligand Expression Vector

An adenoviral vector encoding sig-hAnxA1/CD40L is prepared as follows. Total RNA is extracted from human endothelial cells from a tumor nodule and cDNA was synthesized from total RNA using the SuperScript™ First-Strand Synthesis System (Invitrogen, San Diego, CA). The resulting cDNA is used as the template in the following PCR amplification. The mouse IgG kappa chain METDTLLLWVLLLWVPGSTGD (single letter amino acid code) (SEQ ID NO:21) is prepared by PCR amplification in conjunction with amplification of human annexin A1 to generate the full 21 amino acid mouse IgG kappa chain signal sequence (the start codon “ATG” is shown bolded in SEQ ID NO:22) fused to the N-terminus of the annexin A1 fragment.

| | |
|---|---|
| Primer 1: | |
| 5′-CCACC ATG GAG ACA GAC ACA CTC CTG | (SEQ ID NO:22) |
| CTA TGG GTA CTG CTG-3′; | |
| primer 2: | |
| 5′- TC CTG CTA TGG GTA CTG CTG CTC | (SEQ ID NO:23) |
| TGG GTT CCA GGT TC-3′; | |
| primer 3: | |
| 5′- TG CTC TGG GTT CCA GGT TCC ACT | (SEQ ID NO:76) |
| GGT GAC GCAATG-3′; | |

primer 4 (forward primer for the human annexin A1
fragment):

| | |
|---|---|
| 5′-TCCACTGGTGAC GCAATGGTATCAGAATTCCT | (SEQ ID NO:77) |
| CAAG-3′ | |
| and; and | |

primer 5 (reverse primer for the human annexin A1
fragment):

| | |
|---|---|
| 5′- CGGAGCTCGAG GAGTTTTTAGCAGAGCTAAA | (SEQ ID NO:78) |
| ACAACC -3′. | |

K/AnnexA1 with the upstream kappa signal sequence is generated by four rounds of PCR amplification (1st round: primers 4+5; 2nd round: add primer 3; 3rd round: add primer 2; 4th round: add primer 1). The K/AnnexinA1 encoding DNA is subcloned into the pcDNA™ 3.1 TOPO vector (Invitrogen, San Diego, Calif.) forming pcDNA-K/AnnexinA1(human).

Fragment sp-ΔCtΔTmCD40L(human) is digested with Xba I and Xho I restriction endonucleases and then ligated into pcDNA-K/AnnexinA1(human). The K/AnnexinA1-sp-ΔCtΔTmCD40L fragment is cut from the pcDNA vector and inserted into the pShuttle plasmid using KpnI and Xba I sites (pShuttle K/AnnexinA1-sp-ΔCtΔTmCD40L). Thus, the K/AnnexinA1-sp-ΔCtΔTmCD40L fragment includes the kappa chain secretory signal followed by amino acid residues 1-114 of human annexin A1 followed by a 10 amino acid linker (LENDAQAPKS; SEQ ID NO:29) followed by human CD40 ligand residues.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

1. A method of generating an immune response in an individual against a tumor vascular endothelial cell antigen (TVECA), comprising administering to the individual an effective amount of an expression vector, said vector comprising a transcription unit encoding a secretable fusion protein, said fusion protein comprising the TVECA and CD40 ligand.

2-28. (canceled)

29. A method of treating an individual with cancer having tumor vascular endothelial cells that are specifically associated with a tumor vascular endothelial cell antigen (TVECA), comprising administering to the individual an effective amount of an expression vector, said vector comprising a transcription unit encoding a secretable fusion protein, said fusion protein comprising the TVECA and CD40 ligand.

30-63. (canceled)

64. A nucleic acid encoding a secretable fusion protein, said fusion protein comprising a tumor vascular endothelial cell antigen (TVECA) and CD40 ligand.

65. The nucleic acid of claim 64 wherein said TVECA is selected from the group consisting of annexin A1, annexin A8, VEGF R1, endosialin, and Tie2.

66. The nucleic acid of claim 64 wherein said TVECA are human TVECA.

67. The nucleic acid of claim 64 wherein said TVECA is selected from the group consisting of VEGF R1, endosialin, and Tie2, and wherein said TVECA lack a cytoplasmic domain.

68. The nucleic acid of claim 64 wherein said TVECA is selected from the group consisting of VEGF R1, endosialin, and Tie2, and wherein said TVECA include no more than six residues from either end of the transmembrane domain.

69. The nucleic acid of claim 64 wherein said TVECA is selected from the group consisting of VEGF R1, endosialin, and Tie2, and wherein said TVECA are lacking all or substantially all of a transmembrane domain.

70. The nucleic acid of claim 64 wherein said TVECA is selected from the group consisting of VEGF R1, endosialin, and Tie2, and wherein said TVECA are lacking a transmembrane domain.

71. The nucleic acid of claim 64 wherein said CD40 ligand is human CD40 ligand.

72. The nucleic acid of claim 64 wherein said CD40 ligand lacks a cytoplasmic domain.

73. The nucleic acid of claim 64 wherein said vector encodes a CD40 ligand that includes no more than six residues from either end of the transmembrane domain.

74. The nucleic acid of claim 64 wherein said vector does not encode the transmembrane domain of CD40 ligand.

75. The nucleic acid of claim 64 wherein said CD40 ligand is missing all or substantially all of its transmembrane domain.

76. The nucleic acid of claim 64 wherein said CD40 ligand comprises residues 47-261.

77. The nucleic acid of claim 64 wherein said CD40 ligand comprises residues 1-23 and 47-261.

78. An expression vector comprising the nucleic acid of claim 64.

79. A cell containing the expression vector of claim 78.

80. A protein encoded by the nucleic acid of claim 64.
